(19) United States
(12) Patent Application Publication
(10) Pub. No.: US 2026/0337843 A1
(43) Pub. Date: Oct. 29, 2026

(54) PIN PUNCHED, APERTURED NON-WOVEN WEB AND METHOD OF MAKING SAME

(71) Applicant: FITESA FILM PRODUCTS LLC, Simpsonville, SC (US)

(72) Inventors: Andrew D. MASCHINO, Paris, IL (US); Todd R. SKOCHDOPOLE, Moseley, VA (US); Ben KAHLES, Simpsonville, SC (US)

(21) Appl. No.: 19/657,997

(22) Filed: Apr. 24, 2026

Related U.S. Application Data
(60) Provisional application No. 63/794,803, filed on Apr. 25, 2025.
(60) Provisional application No. 63/813,403, filed on May 28, 2025.

(51) Int. Cl. D04H 3/007; D04H 3/011; D04H 3/105

(52) U.S. Cl. CPC D04H 3/007; D04H 3/011; D04H 3/105; D10B 2401/021; D10B 2401/022; D10B 2509/026

(57) ABSTRACT

A pin punched apertured non-woven web includes a plurality of fibers that define a non-woven web. A second surface is separated from a first surface by a first distance with a plurality of apertures extending through the non-woven web. The plurality of apertures is formed by pressing a plurality of pins, heated within a range of 150-240 °F (65.56-115.56 °C), through the non-woven web. The plurality of apertures define a plurality of proximal sides and distal sides with the proximal sides being located at the first surface. The distal sides define a third surface disposed from the first surface by a second distance. The second distance defines a loft of the non-woven web and is greater than the first distance. The loft is greater than the first distance by 180%-230%.

### CROSS-REFERENCE TO RELATED APPLICATION(S)

This patent application is a United States Non-Provisional Patent Application that relies for priority on U.S. Patent Application Ser. No. 63/794,803, filed on Apr. 25, 2025, and on U.S. Patent Application Ser. No. 63/813,403, filed on May 28, 2025, the contents of both of which are incorporated herein by reference.

### FIELD OF THE INVENTION

The present invention concerns a pin punched, apertured non-woven web that may be employed, in one non-limiting environment, as an acquisition distribution layer (“ADL”) in a disposable, wearable article such as a diaper, adult incontinence garment, or feminine hygiene product. The present invention also encompasses a method of making a pin punched, apertured non-woven web.

### DESCRIPTION OF THE RELATED ART

As should be apparent to those skilled in the art, there are numerous techniques in the art for creating an apertured non-woven material.

Typically, to create a conventional, apertured non-woven, a unapertured non-woven web is mechanically pierced by a plurality of hot pins. The hot pins effectively push through the unapertured non-woven web to create a plurality of holes therethrough.

One downside, among others, with conventional, hot pin punched, non-woven web is that the temperature of the pins and rolls employed in the hot pin aperturing process may be higher than the melting point of the non-woven fibers and, thus, tend to melt the fibers in the non-woven web in the immediate vicinity of the apertures that the hot pins create. Once cooled, the melted fibers coalesce into hard rings around the apertures. These hard rings are tactilely unsatisfactory. Moreover, they present an unappealing visual appearance to the hot pinned, apertured non-woven web.

Among other objectives, the present invention seeks to avoid the creation of these hard rings.

Separately, it is noted that traditional hot pin punching does not alter the loft, or thickness, of the non-woven web, although there may be some minor distortions created at the locations of the hard rings resulting from the pushing of the pins through the non-woven web.

The present invention seeks to create an apertured non-woven web with a loft higher than the precursor, apertured non-woven web. This includes creating an apertured non-woven web with a loft higher than those created via traditional hot pin approaches.

Still further advantages and features of the present invention will be made apparent by the discussion presented hereinbelow.

### SUMMARY OF THE INVENTION

The present invention seeks to address one or more of the deficiencies in the prior art.

Without limiting the present invention, the present invention seeks to create a pin punched, apertured non-woven web having a plurality of fibers, a first surface defining a garment facing side to non-woven web, and a second surface defining a user facing side to non-woven web. The second surface is separated from the first surface by a first distance. The pin punched, apertured non-woven web also includes a plurality of apertures extending through the non-woven web from the first surface to the second surface, where the plurality of apertures is formed by pressing a plurality of pins, heated within a range of 150-240 °F (65.56-115.56 °C), through the non-woven web, causing the plurality of pins to punch through the non-woven web. The plurality of apertures define a plurality of proximal sides and distal sides, where the proximal sides are located at the first surface. The distal sides define a third surface disposed from the first surface by a second distance. The second distance defines a loft of the non-woven web. The second distance is greater than the first distance, and the loft is greater than the first distance within a range of 180%-230%.

In at least one contemplated embodiment, a plurality of bond points secure the plurality of fibers to one another to define a non-woven web. The plurality of bond points encompass at least one of ultrasonic welds, thermal bonds, pressure bonds, mechanically deformed bonds, resin bonds, adhesive bonds, and bonds between individual fibers.

In a selected embodiment, it is contemplated that the distal sides of the plurality of apertures comprise edge portions that differ from ideal edge perimeters, defining an aperture fidelity within a range of 0.80-1.00, within a range of 0.85-0.95, within a range of 0.87-0.93, within a range of 0.89-0.91, or about 0.90.

In other contemplated embodiments, the non-woven web has a first open area without the plurality apertures and a second open area with the plurality of apertures, the second open area is greater than the first open area, and a ratio of the first open area to the second open area is within a range of 1.5-9.0, within a range of 4.0-5.0, within a range of 4.3-4.7, within a range of 4.4-4.6, or about 4.5.

Still further, it is contemplated that the loft is greater than the first distance within a range of 190%-210%, within a range of 195%-205%, or by about 200%.

In one or more embodiments, the plurality of fibers includes polyethylene terephthalate, poly lactic acid, polyethylene, polyethylene and polyethylene terephthalate, polypropylene, polyethylene and polypropylene, polyethylene and polylactic acid, and/or polyethylene terephthalate and co-polyethylene terephthalate.

In one or more contemplated embodiments, the pin punched apertured non-woven web comprises a plurality of fibers having a weight from 1 to 10 Den.

In other embodiments, the plurality of fibers with a glass transition temperature between 5° and 85 °C (122-185 °F), wherein the plurality of fibers include at least one material.

The present invention also provides an absorbent product. The absorbent product includes a pin punched apertured non-woven web, an absorbent core disposed adjacent to the pin punched apertured non-woven web, and a back sheet disposed adjacent to the absorbent core. The absorbent core is sandwiched between the pin punched apertured non-woven web and the backsheet. The pin punched apertured non-woven web includes a plurality of fibers defining a non-woven web, a first surface defining a garment facing side to non-woven web, a second surface defining a user facing side to non-woven web, wherein the second surface is separated from the first surface by a first distance, and a plurality of apertures extending through the non-woven web from the first surface to the second surface. The plurality of apertures is formed by pressing a plurality of pins, heated within a range of 150-240 °F (65.56-115.56 °C), through the non-woven web, causing the plurality of pins to punch through the non-woven web. The plurality of apertures define a plurality of proximal sides and distal sides. The proximal sides are located at the first surface. The distal sides define a third surface disposed from the first surface by a second distance, where the second distance defines a loft of the non-woven web, the second distance is greater than the first distance, and the loft is greater than the first distance within a range of 180%-230%.

In a contemplated embodiment, the absorbent product also includes a topsheet disposed adjacent to the pin punched apertured non-woven web, where the pin punched apertured non-woven web is sandwiched between the topsheet and the back sheet.

The absorbent product incorporating the pin punched apertured non-woven web is contemplated to embody one or more characteristics of the pin punched apertured non-woven web as discussed in greater detail herein.

Still further, the present invention encompasses a pin punched apertured non-woven web that includes a first non-woven web and a second non-woven web. The first and second non-woven webs are similarly constructed to one another. Each are constructed from a plurality of fibers with a first surface defining a garment facing side and a second surface defining a user facing side. The second surface is separated from the first surface by a first distance. A plurality of apertures extending through the first non-woven web from the first surface to the second surface. The plurality of apertures is formed by pressing a plurality of pins, heated within a range of 150-240 °F (65.56-115.56 °C), through the first and second non-woven webs, causing the plurality of pins to punch through the first and second non-woven webs. The plurality of apertures define a plurality of proximal sides and distal sides, where the proximal sides are located at the first surface and the distal sides define a third surface disposed from the first surface by a second distance. The second distance defines a loft of the first and second non-woven webs. The second distance is greater than the first distance. The loft is greater than the first distance within a range of 180%-230%. The first non-woven web is secured to the second non-woven web by at least the plurality of pins being punched therethrough.

In one embodiment, the first non-woven web is hydrophobic and the second non-woven web is hydrophilic.

In another embodiment, the first non-woven web is hydrophilic and the second non-woven web is hydrophobic.

The present invention also encompasses an absorbent article that includes a multi-layer pin-punched apertured non-woven web together with an absorbent core disposed adjacent to the pin punched apertured non-woven web and a back sheet disposed adjacent to the absorbent core, wherein the absorbent core is sandwiched between the pin punched apertured non-woven web and the backsheet.

Still other aspects and features of the present invention will be made apparent from the description that follows.

### BRIEF DESCRIPTION OF THE DRAWINGS

The present invention will now be described in connection with the drawings appended hereto, in which:

FIG. 1 is a graphical, cross-sectional side view of an absorbent product incorporating the pin punched, apertured non-woven according to the present invention;

FIG. 2 is a graphical, cross-sectional side view of a non-woven precursor web prior to being pin punched in accordance with the present invention;

FIG. 3 is graphical, cross-sectional side view of the non-woven precursor web illustrated in FIG. 2, immediately prior to pin punching in accordance with the present invention;

FIG. 4 is a graphical, cross-sectional representation of the non-woven precursor web illustrated in FIGS. 2 and 3, showing one aperture formed in the non-woven web after being pin punched to form a pin punched, apertured non-woven web in accordance with the present invention;

FIG. 5 is a graphical top view of a representative example of one aperture in a non-woven web after being pin punched in accordance with the present invention via a star-shaped pin;

FIG. 6 is a graphical representation of the definition of aperture integrity for the apertures formed in accordance with the present invention;

FIG. 7 is a graphical illustration providing an overview of one contemplated apparatus for manufacturing the pin punched, apertured non-woven web according to the present invention;

FIG. 8 is an enlarged photograph of the garment facing side of a non-woven web made from a combination of polyethylene (“PE”) and polyethylene terephthalate (“PET”) (also referred to as “PE/PET”), illustrating a plurality of apertures after having been pin punched, with star-shaped pins, at 180 °F (82.22 °C) in accordance with the present invention;

FIG. 9 is a first, enlarged photograph of the user facing side of the non-woven web shown in FIG. 8, where the photograph is taken at an angle to the user facing side of the non-woven web to highlight details with respect to the plurality of apertures formed therein by pin punching, with star-shaped pins, at 180 °F (82.22 °C) in accordance with the present invention;

FIG. 10 is a second, enlarged photograph of the user facing side of the non-woven web shown in FIGS. 8 and 9 where the photograph is taken at a different angle to the angle of the photograph provided in FIG. 9 to highlight additional details with respect to the plurality of apertures formed therein by pin punching, with star-shaped pins, at 180 °F (82.22 °C) according to the present invention;

FIG. 11 is an enlarged photograph of the garment facing side of a non-woven web made from a combination of polyethylene (“PE”) and polypropylene (“PP”) (also referred to as “PE/PP”), illustrating a plurality of apertures after having been pin punched at 180 °F (82.22 °C) in accordance with the present invention;

FIG. 12 is an enlarged photograph of the non-woven web shown in FIG. 10, where the photograph is taken at an angle to the user facing side of the non-woven web to highlight details with respect to the plurality of apertures formed therein by pin punching at 180 °F (82.22 °C) in accordance with the present invention;

FIG. 13 is an enlarged photograph of the garment facing side of a non-woven web made from a combination of polyethylene (“PE”) and polypropylene (“PP”) (also referred to as “PE/PP”), illustrating a plurality of apertures after having been pin punched at 220 °F (104.44 °C) in accordance with the present invention;

FIG. 14 is an enlarged photograph of the non-woven web shown in FIG. 13, where the photograph is taken at an angle to the user facing side of the non-woven web to highlight details with respect to the plurality of apertures formed therein by pin punching at 220 °F (104.44 °C) in accordance with the present invention;

FIG. 15 is an enlarged photograph of the garment facing side of a non-woven web made from crimped polypropylene (“PP”) fiber, illustrating a plurality of apertures after having been pin punched at 220 °F (104.44 °C) in accordance with the present invention;

FIG. 16 is a first, enlarged photograph of the non-woven web shown in FIG. 15, where the photograph is taken at an angle to the user facing side of the non-woven web to highlight details with respect to the plurality of apertures formed therein by pin punching at 220 °F (104.44 °C) in accordance with the present invention;

FIG. 17 is a second, enlarged photograph of the non-woven web shown in FIGS. 15 and 16 where the photograph is taken at a different angle to the angle of the photograph provided in FIG. 16 to highlight details with respect to the plurality of apertures formed therein by pin punching at 220 °F (104.44 °C) in accordance with the present invention;

FIG. 18 is an enlarged photograph of the garment facing side of a non-woven web made from PE/PET, illustrating a plurality of apertures after having been pin punched, by diamond-shaped pins, at 150 °F (65.56 °C) in accordance with one embodiment of the present invention;

FIG. 19 is an enlarged photograph of the user facing side of the non-woven web shown in FIG. 18 where the photograph is taken at an angle to highlight details with respect to the plurality of apertures formed therein by pin punching, by diamond-shaped pins, at 150 °F (65.56 °C) in accordance with the present invention;

FIG. 20 is an enlarged photograph of the garment facing side of a non-woven web made from PE/PET, illustrating a plurality of apertures after having been pin punched, by diamond-shaped pins, at 240 °F (115.56 °C) in accordance with one embodiment of the present invention;

FIG. 21 is an enlarged photograph of the user facing side of the non-woven web shown in FIG. 20 where the photograph is taken at an angle to highlight details with respect to the plurality of apertures formed therein by pin punching, by diamond-shaped pins, at 240 °F (115.56 °C) in accordance with the present invention;

FIG. 22 is an enlarged photograph of the garment facing side of a non-woven web made from PP fiber, illustrating a plurality of apertures after having been pin punched, by diamond-shaped pins, at 240 °F (115.56 °C) in accordance with one embodiment of the present invention;

FIG. 23 is an enlarged photograph of the user facing side of the non-woven web shown in FIG. 22 where the photograph is taken at an angle to highlight details with respect to the plurality of apertures formed therein by pin punching, by diamond-shaped pins, at 240 °F (115.56 °C) in accordance with the present invention;

FIG. 24 is a graphical, cross-sectional side view of another embodiment of an absorbent product combining two non-woven webs to form a pin punched, apertured non-woven according to the present invention;

FIG. 25 is a graphical, cross-sectional side view of one further embodiment of an absorbent product combining two non-woven webs to form a pin punched, apertured non-woven according to the present invention;

FIG. 26 is a graphical, cross-sectional side view of an additional embodiment of an absorbent product combining two non-woven webs to form a pin punched, apertured non-woven according to the present invention;

FIG. 27 is a graphical, cross-sectional side view of an embodiment of an absorbent product combining three non-woven webs to form a pin punched, apertured non-woven according to the present invention;

FIG. 28 is a graphical, cross-sectional side view of still one more embodiment of an absorbent product combining three non-woven webs to form a pin punched, apertured non-woven according to the present invention;

FIG. 29 is a graphical, cross-sectional side view of two non-woven precursor webs prior to being pin punched in accordance with the present invention;

FIG. 30 is graphical, cross-sectional side view of the two non-woven precursor webs illustrated in FIG. 29, immediately prior to pin punching in accordance with the present invention; and

FIG. 31 is a graphical, cross-sectional representation of the two non-woven precursor webs illustrated in FIGS. 29 and 30, showing one aperture formed in the two non-woven webs after being pin punched to form a pin punched, apertured non-woven web in accordance with the present invention.

### BRIEF DESCRIPTION OF EMBODIMENT(S) OF THE INVENTION

The present invention will now be described in connection with one or more embodiments. Where possible, the same reference numbers are employed to refer to like structures and/or features. Unless otherwise indicated, the use of the same reference numbers should not be understood to mean that each structure using the same reference number is identical to each other structure using that reference number. To the contrary, as should be apparent to those skilled in the art, variations and equivalents of the structures may be employed. Those variations and equivalents are contemplated to be encompassed by the present invention, even if not explicitly discussed herein.

The present invention also will be described in connection with one or more materials. Any materials described herein are intended to be exemplary of the possible materials that may be employed and are not intended to limit the scope of the present invention. Moreover, any discussion about specific characteristics and parameters of any material should not be understood to be limiting of the material, unless otherwise stated.

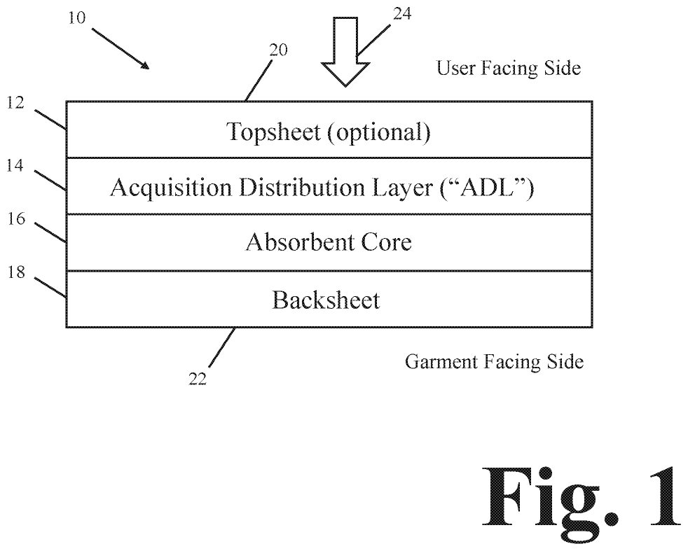

FIG. 1 is a graphical, side view representation of an absorbent product 10 according to the present invention. The absorbent product 10 includes a topsheet 12, an acquisition distribution layer (“ADL”) 14, an absorbent core 16, and a backsheet 18.

It is noted that the topsheet 12 is an optional layer, as discussed hereinbelow.

The absorbent product 10 illustrated in FIG. 1 may be employed in any of a number of products that are designed to capture and secure one or more human exudates. Exudates include, but are not limited to urine, feces, blood, etc. Non-limiting examples of products that may incorporate the absorbent product 10 include feminine hygiene products, diapers for infants, adult diapers, incontinence products, and the like.

As illustrated in FIG. 1, the absorbent product 10 is defined by a user facing side 20 and a garment facing side 22. The user facing side 20 faces the user, as the name suggests. Specifically, the user facing side 20 of the absorbent product 10 is contemplated to be in direct contact with a user's skin. Conversely, the garment facing side 22 of the absorbent product 10 is designed to be in contact with the user's clothing or garments.

As should be apparent to those skilled in the art, exudates (hereinafter referred to as “insults”) are introduced to the absorbent product 10 in the direction of the arrow 24.

Insults that are introduced to the absorbent product 10 first encounter the topsheet 12, which may be a porous film that permits the insult to pass therethrough. The topsheet 12 is an optional component of the absorbent product 10. Depending on the type of insult and the construction of the absorbent product 10, the topsheet 12 may not be desired or required.

From the topsheet 12, the insult passes to the ADL 14. As the name suggests, the ADL 14 receives the insult and distributes the insult across a broader area from the location where the insult originates.

From the ADL 14, the insult passes to the absorbent core 16. The absorbent core 16 comprises a material, such as a hydrophilic gel, that captures and fixes the insult therein.

The backsheet 18 is a fluid impervious layer that prevents the insult from migrating to the user's garments.

In several of the figures, the designations “User Facing Side” and “Garment Facing Side” are designated to facilitate discussion of various aspects of the present invention. As noted above, the user facing side also is designated “20” and the garment facing side is designated “22.”

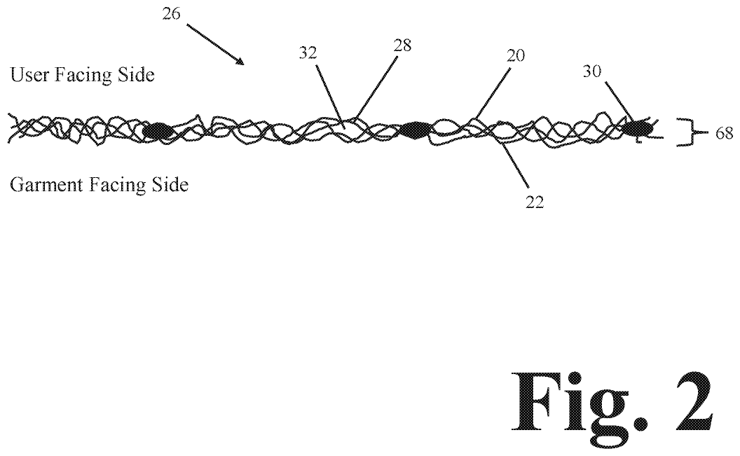

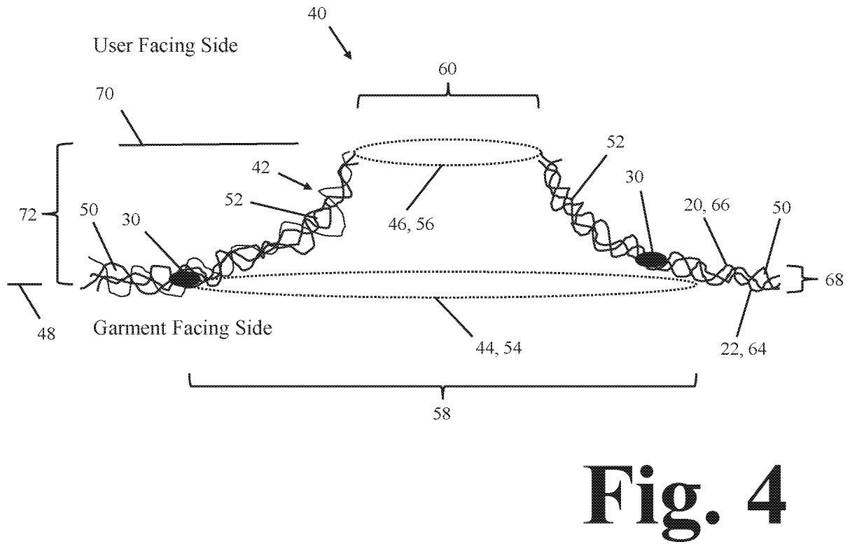

FIG. 2 is a graphical, cross-sectional illustration of a non-woven precursor web 26 of the type that may be employed to create the pin punched, apertured non-woven web 40 (as illustrated in FIG. 4) according to the present invention. In this view, the non-woven precursor web 26 is made from a plurality of fibers 28 that are connected to one another via a plurality of bond points 30. The bond points 30 may be created via ultrasonic welding, thermal bonding, pressure bonding, mechanical deformation, resin bonds, adhesive bonds, bonds between individual fibers, and the like during the manufacture of the non-woven precursor web 26. As should be apparent to those skilled in the art, the non-woven precursor web 26 includes voids 32 between the fibers 28. The construction of the unapertured non-woven precursor web 26 is well known to those skilled in the art and, therefore, further elaboration is not provided here.

While FIG. 2 illustrates what those skilled in the art would understand to be a spun bonded non-woven for the non-woven precursor web 26, the invention is not intended to be limited thereto. For example, the non-woven precursor web 26 may be any type of carded web including, but not limited to, a resin bonded non-woven web, a multi-bonded non-woven web, a thermal bonded non-woven web, a spun lace web, and/or an air-through bonded (“ATB”) non-woven web.

In the case of the ATB non-woven web, hot air is forced through the fibers so that adjacent fibers in the web melt and adhere to one another, thereby forming bonds between individual fibers in the web. The individual fiber bonds in an ATB non-woven web are subsumed within the definition of the bonds 30 illustrated in FIG. 2, as should be apparent to those skilled in the art.

It is noted that the depiction of the bond points 30 is merely intended to be exemplary of one or more of the non-woven webs that may be employed (e.g., a thermal bonded non-woven web or an ATB non-woven web). Bonds 30 are not required to practice the invention, as should be apparent to those skilled in the art.

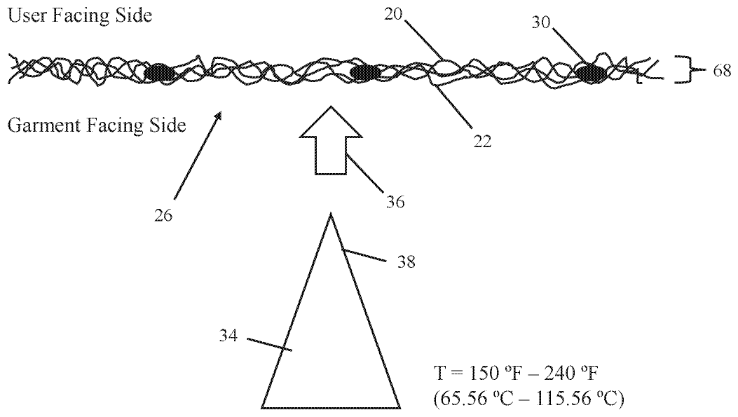

FIG. 3 is graphical, cross-sectional side view of the non-woven precursor web 26 illustrated in FIG. 2, immediately prior to pin punching in accordance with the present invention.

To create the pin-punched non-woven web 40 according to the present invention, a pin 34 is pressed through the non-woven precursor web 26 in the direction of the arrow 36 until the tip 38 of the pin 34 passes through the non-woven precursor web 26.

As illustrated, the pin 34 is pressed into the non-woven precursor web 26 from the garment facing side 22. Some portion of the tip 38 of the pin 34 is contemplated to project out from the user facing side 20 when the pin 34 is at a desired penetration of the non-woven precursor web 26 to form the pin-punched non-woven web 40 according to the present invention.

FIG. 4 is a graphical, cross-sectional representation of the pin-punched non-woven web 40 according to the present invention.

One aperture 42 in pin-punched non-woven web 40 is shown. The aperture 42 is representative of the plurality of apertures 42 that are contemplated to be formed in the pin-punched non-woven web 40.

As illustrated in FIG. 4, the aperture 42 includes a proximal side 44 and a distal side 46. The proximal side 44 is coplanar or nearly coplanar with a plane 48 defined be the lands 50. The lands are the portions of the pin-punched non-woven web 40 that are coplanar or nearly coplanar with the plane 48, between the apertures 42.

As also shown in FIG. 4, the aperture 42 has a generally conical shape (or volcano shape), which is defined by curved walls 52 extending from the proximal side 44 to the distal side 46 of the aperture 42. It is noted that while reference is made to two curved walls 52 as a convenience, the curved walls 52 also may be considered as a single wall extending in the user facing direction.

The curved walls 52 establish the generally conical shape of the aperture 42. Being generally conical in shape, the aperture has a proximal area 54 that is larger than a distal area 56.

In FIG. 4, for simplicity, the aperture 42 is illustrated with a circular shape. As such, the proximal area 54 defines a circle. Similarly, the distal area 56 also defines a circle. As also illustrated in FIG. 4, the proximal area 54 has a proximal width 58. The distal area 56 has a distal width 60. The aperture 42 defines a height 72, which defines a height distance between the proximal side 44 and the distal side 46 of the aperture 42. The height 72 also is referred to as the loft of the pin punched, apertured non-woven web 40.

With continued reference to FIG. 4, the garment facing side 22 of the pin punched, apertured non-woven web 40 defines a first surface 64. The user facing side 20 of the pin punched, apertured non-woven web 40 defines a second surface 66. The first surface 64 is separated from the second surface 66 by a thickness 68, which is the thickness (or loft) of the non-woven precursor web 26.

The proximal sides 44 of the apertures 42 are located at the first surface 64 on the garment facing side 22. The distal sides 46 of the apertures 42 define a third surface 70 that is separated from the first surface 64 by a second distance that is the thickness 72, or loft, of the pin punched, apertured non-woven web 40.

As illustrated in FIG. 4, and as discussed in greater detail herein, the height or loft 72 of the pin punched, apertured non-woven web 40 is greater than the thickness or loft 68 of the non-woven precursor web 26. More specifically, the pin punched loft 72 is greater than the precursor loft 68 within a range of 180%-230%.

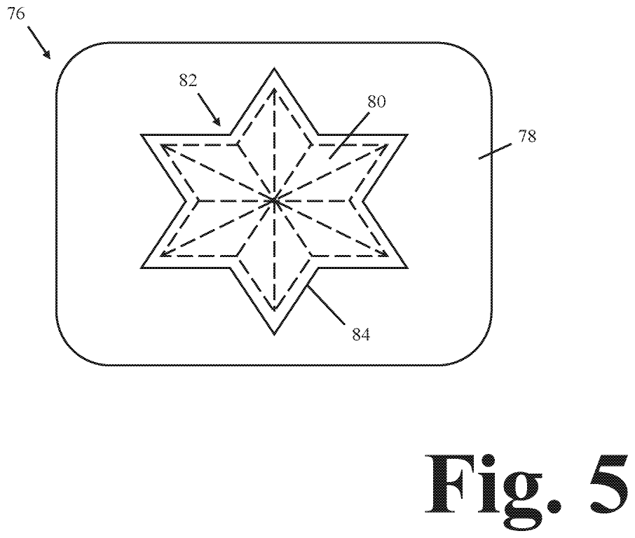

FIG. 5 is a graphical top view of a representative example of one aperture 82 in a pin punched, apertured non-woven web 76 after being pin punched into the non-woven web 78 accordance with the present invention via a star-shaped pin 80. Having been punched with a star-shaped pin 80, the aperture 82 has an edge 84 that is star-shaped.

In this view, as with FIGS. 2-4, only one aperture 82 is shown. This aperture 82 is contemplated to be representative of all of the apertures 82 in the pin-punched non-woven web 78.

In connection with FIG. 5, it is contemplated that all of the apertures 82 in the pin punched, apertured non-woven web 76 will have the same shape. However, the present invention should not be understood to be limited solely to such a configuration. It is contemplated that the pin punched, apertured non-woven web 76 may include apertures 82 will differing shapes. In one non-limiting example, a central location in the pin punched, apertured non-woven web 76 may have apertures 82 with a star shape, while surrounding apertures 82 may be circular in shape.

As discussed in greater detail herein, one aspect of the pin-punched, apertured non-woven web 40, 76 of the present invention lies in the creation of an identifiable edge 84 for the apertures 42, 82. Specifically, the pin-punched, apertured non-woven web 40, 76 of the present invention is characterized in that the edge 84 of the aperture 42, 82 retains a high fidelity with respect to the shape of the pin 34, 80 employed to create the apertures 42, 82.

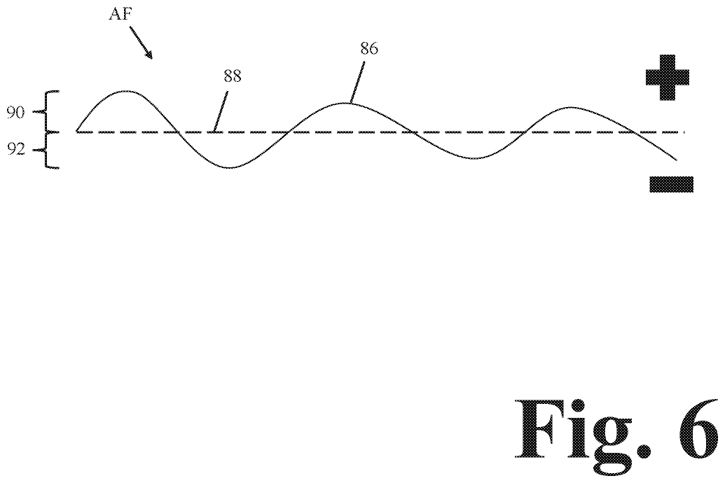

FIG. 6 illustrates one way in which fidelity AF of the apertures 42, 82 may be evaluated in connection with the present invention. It is noted that this evaluation methodology is merely exemplary of one of several methods that may be employed to measure aperture fidelity AF.

FIG. 6 illustrates an edge portion 86 of an aperture 42, 82. To simplify the discussion of aperture fidelity AF, the edge portion 86 is contemplated to be a portion of the edge 84 that is illustrated for the aperture 82 shown in FIG. 5.

In FIG. 6, the edge portion 86 is illustrated with a wavy line to indicate that the edge portion 86 deviates from the ideal edge perimeter 88 identified by a dotted line. As shown, the edge portion 86 deviates from the ideal edge perimeter 88 by a positive variance 90 or by a negative variance 92. The variance of the edge portion 86 from the ideal edge perimeter 88 defines the concept of fidelity as related to the formation of the aperture 42, 82.

With continued reference to FIG. 6, the aperture fidelity AF is defined with respect to the distal side 46 of the aperture 42, 82. In particular, it is desirable for the distal side 46 of the aperture 42, 82 to follow or match the shape of the pin 34, 80 as closely as possible. This permits the manufacturer to create apertures 42, 82 with any of a number of shapes.

As should be apparent to those skilled in the art, the shape of each aperture 42, 82 at both the distal side 46 and at the proximal side 44 may impact on the transmissibility of insults through the ADL 14 from the user facing side 20 to the absorbent core 16 on the garment facing side 22. Specifically, it is contemplated that selected shapes for the apertures 42, 82 may help to establish a greater transmissibility than other shapes, even though both shapes may have identical open areas 54, 56. It is noted that, because the proximal and distal areas 54, 56 refer to the areas of the openings of the proximal side 44 and of the distal side 46 of the apertures 42, 82, these areas also are referred to herein as “open areas” 54, 56 herein.

The shape of the aperture 42, 82 at both the distal side 46 and the proximal side 44 also may impact on a property known as “rewet.” Rewet refers to the transfer of fluids from the garment facing side 22 of the ADL 14 to the user facing side 20. Rewet, therefore, refers to the travel of fluids in a direction opposite to the desired direction, which is to the absorbent core 16.

With renewed reference to FIG. 6, it is contemplated that the distal open area 56 defined by the edge 84 of the aperture 42, 82 may be measured from a center point or an alternative locus. For example, where the pin 34, 80 is conical, the open proximal area 54 and the open distal area 56 are both contemplated to be circular. The distance to a center point is the radius of the circle defining the open distal area 56.

If the edge 84 illustrated in FIG. 6 has an AF of 1.00, this would indicate that the aperture fidelity AF is 100%. In such a case, the edge 84 would be colinear with the ideal edge perimeter 88. In the case illustrated in FIG. 6, the AF is not 1.00 (or 100%). Instead, the positive variation 90 and the negative variation 92 define a deviation from the ideal edge perimeter 88. In the illustrated example, the deviation is ±5% (or 0.05), which results in a fidelity AF of 0.95 (or 95%).

For purposes of the present invention, it is contemplated that the apertures 42, 82 will have an aperture fidelity AF of greater than 0.80 (80%). Ranges for the aperture fidelity AF are contemplated to be defined by any of the integer values from 80% to 100% (0.80-1.00). As a result, any range or value defined by endpoints, inclusive thereof, from 0.80 (80%) to 1.00 (100%) for the aperture fidelity AF is contemplated to fall within the scope of the present invention. This includes aperture fidelities AF of 0.80 (80%), 0.81 (81%), 0.82 (82%), 0.83 (83%), 0.84 (84%), 0.85 (85%), 0.86 (86%), 0.87 (87%), 0.88 (88%), 0.89 (89%), 0.90 (90%), 0.91 (91%), 0.92 (92%), 0.93 (93%), 0.94 (94%), 0.95 (95%), 0.96 (96%), 0.97 (97%), 0.98 (98%), 0.99 (99%), and 1.00 (100%). One exemplary range includes, but is not limited to, 0.82-0.90.

With respect to the aperture fidelity AF, it is contemplated that the apertures 42, 82 will be provided with an average aperture fidelity of 0.90. In particular, contemplated ranges for the aperture fidelity AF include 0.85-0.95, 0.87-0.94, and 0.89-0.91.

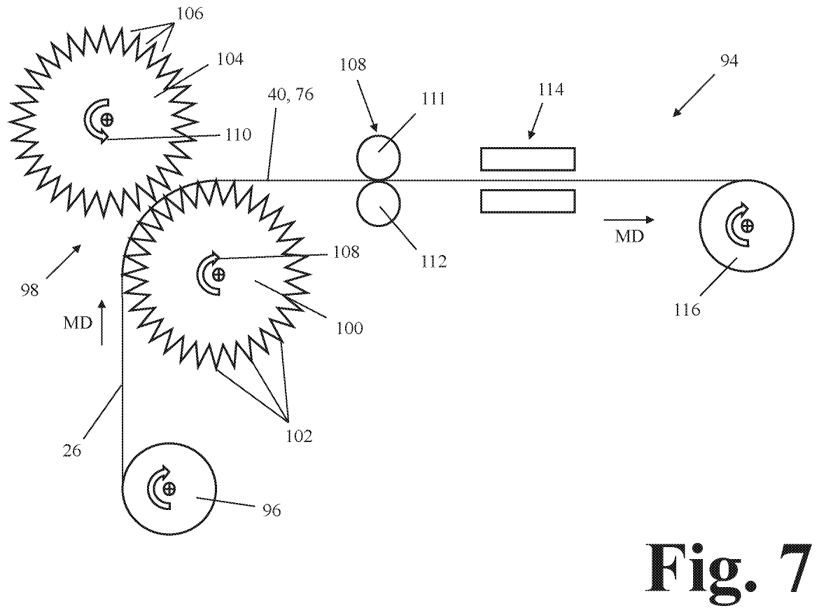

FIG. 7 is a graphical representation of pin punching apparatus 94 that may be employed to manufacture the pin punched, apertured non-woven web 40, 76 according to the present invention.

As shown, the non-woven precursor web 26 may be unwound from a first roller 96. Alternatively, the non-woven precursor web 26 may be manufactured in situ and fed directly into the apparatus 94 after being manufactured. Since the manufacture of the non-woven precursor 26 is within the level of skill in the art, details concerning the manufacture of the non-woven precursor web 26 are not provided here.

The non-woven precursor web 26 (see FIG. 2) is conveyed, under tension, to a pin punching station 98. For reference, the direction of travel of the unapertured non-woven precursor web 26 is the machine direction MD, as shown. It is noted that the cross-direction, which is across the width of the unapertured non-woven precursor web 26, is the transverse direction TD. The transverse direction TD is orthogonal to the machine direction MD, as should be apparent to those skilled in the art. The transverse direction TD defines, in part, the width of the non-woven precursor web 26 passing through the pin punching apparatus 94.

The pin punching station 98 includes a first pinned roller 100 with a plurality of pins 102 disposed on its surface (also referred to as a “male” roller). In one contemplated embodiment, the pins 102 are separated from one another so that there is a space or “valley” between adjacent pins 102. In another contemplated embodiment, one or more of the pins 102 are in contact with one or more adjacent pins 102, as required or as desired. In other words, the spacing of the pins 102 from one another may be varied without departing from the scope of the present invention.

The pin punching station 98 also includes a second roller 104 that includes a plurality of openings 106 therein (also referred to as a “female” roller). The openings 106 are complimentary to the pins 102 such that a pin 102 becomes disposed, at least partially, within an opening 106 to form the apertures 42, 82.

As illustrated in FIG. 7, the rollers 100, 104 are counter-rotating rollers, as indicated by the arrows 108, 110.

From the pin punching apparatus 98, the pin punched, apertured non-woven web 40, 76 may proceed to other stations. For example, as illustrated in FIG. 7, the pin punched, apertured non-woven web 40, 76 may proceed to an embossing station 108 (optional).

At the embossing station 111, the pin punched, apertured non-woven web 40, 76 may be passed through a third and fourth roller 113, 112, which apply an embossed design to the pin punched, apertured non-woven web 40, 76. Since embossing is known to those skilled in the art, further details are not provided here.

After the embossing station 98, the pin punched, apertured non-woven web 40, 76 may proceed to further stations for processing. In FIG. 7, a coating station 114 is illustrated. Here, a treatment or coating may be applied, optionally, to the pin punched, apertured non-woven web 40, 76 as required or as desired.

The pin punched, apertured non-woven web 40, 76 is then wound onto a fifth roller 116.

It is noted that the pin punching apparatus 94 is provided as one, non-limiting example of an apparatus that may be employed to manufacture the pin punched, apertured non-woven web 40, 76 of the present invention. The present invention, however, should not be understood to be limited to this example.

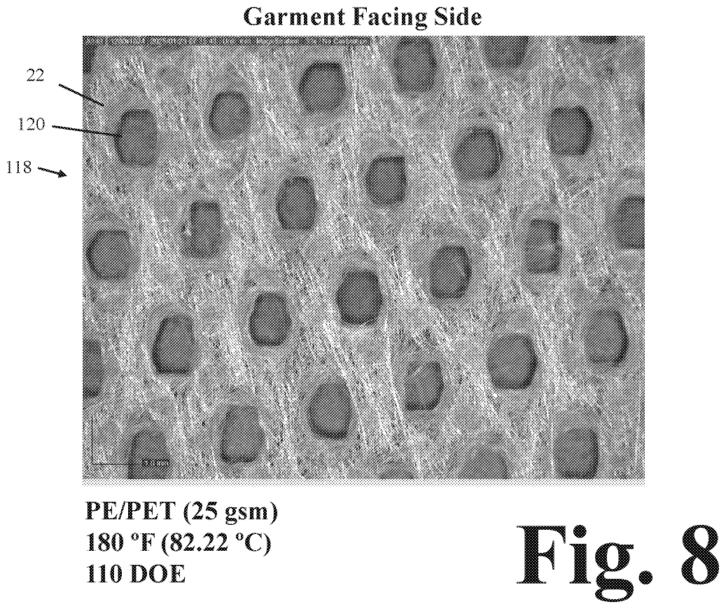

FIG. 8 is an enlarged photograph of one embodiment of a pin punched, apertured non-woven web 118 according to the present invention. Specifically, FIG. 8 shows the garment facing side 22 of the pin punched, apertured non-woven web 118. The pin punched, apertured non-woven web 118 includes a plurality of apertures 120 that have a star shape, having been punched via a plurality of star shaped pins 80 illustrated in FIG. 5, for example.

The pin punched, apertured non-woven web 118 is contemplated to be made from a combination of polyethylene (“PE”) and polyethylene terephthalate (“PET”). This combination is referred to herein as “PE/PET.” In this figure, the pin punched, apertured non-woven web 118 had a basis weight of 25 grams per square meter (“gsm”).

As is apparent from FIG. 8, the apertures 120 have well-defined shapes that are consistent with a star-shaped pin 80. This illustrates, among other aspects, that the apertures 120 have a high aperture fidelity AF within a range of 0.80-1.00, as discussed hereinabove.

To appreciate other aspects of the pin punched, apertured non-woven web 118, the Applicant provides Table 1, below. Table 1 compares selected aspects of the pin punched, apertured non-woven web 118 with the non-woven precursor web 26 prior to pin punching.

TABLE 1

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 25 gsm PE/PET | 168 | 141 | 2.9 | 10.1 | 4.3 |
| Non-woven | | | | | |
| Star Pin Punched 25 | 650 | 281 | 12.8 | 12.1 | 4.6 |
| gsm PE/PET Non- | | | | | |
| woven (180 °F | | | | | |
| (82.22 °C)) | | | | | |
| (110 DOE) | | | | | |

In Table 1, the Embossed Thickness (μm) refers to the thickness of the non-woven material. Here, the unapertured, non-woven precursor web 26 had a thickness of 168 μm. This value is also referred to as the “loft” of the material, as noted above. After pin punching, the pin punched, apertured non-woven web 118 has an embossed thickness (or loft) of 650 μm. For reference, the thickness/loft 72 of the aperture 42, shown in FIG. 4, is the same parameter as the embossed thickness (or loft) reported in Table 1.

“Air Permeability” refers to how quickly air passes through the non-woven material. As provided in Table 1, the unapertured, non-woven precursor web 26 had an air permeability of 141 m³/m²/min. After being pin punched, the air permeability of the pin punched, apertured non-woven web 118 increased to 281 m³/m²/min.

The “Open Area” refers to the open area between the fibers 28 of the non-woven material. The unapertured, non-woven precursor web 26 had an open area of 2.9%. After being pin punched, the pin-punched, apertured non-woven web 118 had an open area of 12.8%.

As may be evident from the photograph provided in FIG. 8, for example, due to the formation of the apertures 120, the non-woven precursor web 26 will have a first open area that is less than the second open area of the pin-punched, apertured non-woven web 118. Simply, the apertures 120 establish a larger open area after pin punching.

For purposes of the present invention, it is contemplated that the relationship between the first open area and the second open area assists with control over the degree of transmissibility of insults through the pin-punched, apertured non-woven web 42, 76. This relationship also assists with control over rewet.

As noted herein, higher transmissibility is desirable, because higher transmissibility translates into a faster transmission of insults through the ADL 14. Lower rewet is desirable, because lower rewet results in an absorbent product 10 that has a drier feel. Transmissibility and rewet are, in many instances variables that are in conflict with one another. As a general rule a higher transmissibility often leads to a higher rewet. Accordingly, so that it is possible to manufacture an absorbent product 10 that user's find desirable, properties of transmissibility and rewet are balanced.

Since the open area of the ADL 14 satisfies both the properties of transmissibility and rewet, it is contemplated that the relationship between the first open area and the second open area are controlled to establish a suitable ratio for these parameters. Accordingly, it is contemplated that a ratio between the first open area and the second open area lies within a range of 1.5-9.0.

For the embodiment illustrated in FIG. 8, it is noted that the star-shaped pins 80 pierced the unapertured, precursor non-woven web at 180 °F or 82.22 °C.

The pin punched, apertured non-woven web 118 exhibited a strikethrough of 4.75 seconds. In the art, faster strikethrough times are preferred, because this indicates that any fluid insults quickly pass more quickly through the ADL 14 to the absorbent core 16. Accordingly, lower strikethrough values are preferred to higher strikethrough values.

In addition, the pin punched, apertured non-woven web 118 had a rewet of 0.09 grams. A higher rewet value indicates a smaller amount of fluid that travels from the absorbent core 16 through the ADL 14. Accordingly, higher rewet values are preferred over lower rewet values.

“Handle” (or “fabric handle”) is a term used by those skilled in the art to quantify the feel of the material. As such, handle depends upon the sense of touch. Different types of material will have different degree of smoothness or roughness when referring to the “handle.” Here, the “handle” was measured by the Handle-O-Meter from the Thwing-Albert Instrument Company (Part #211-2005). The “handle” is measured in grams, as should be apparent to those skilled in the art. As a general rule, a higher handle is preferred over a lower handle, as should be apparent to those skilled in the art.

With continued reference to FIG. 8, it is noted that the pin punched, apertured non-woven web 118 was pin punched to a Depth of Engagement (“DOE”) of 110, which refers to a depth of engagement of 0.110 inches (2.79 mm) for the pins penetrating the non-woven precursor web 26.

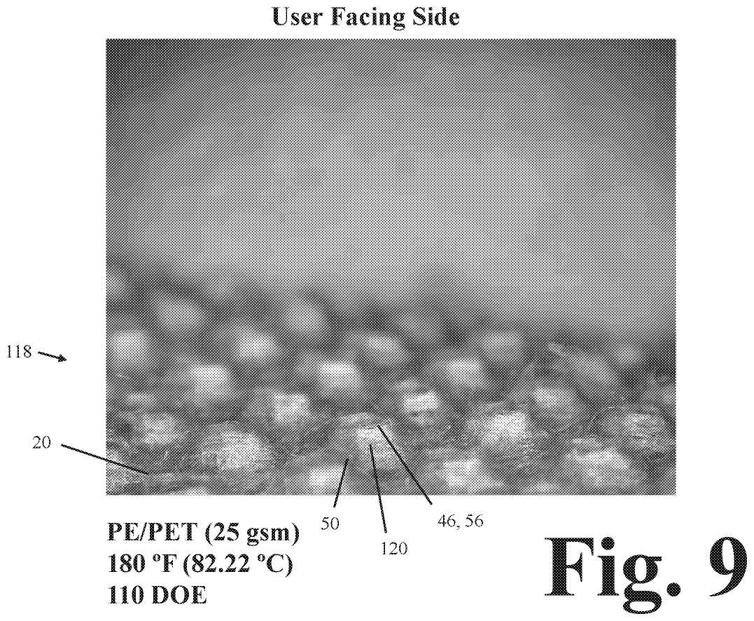

FIG. 9 is a first, enlarged photograph of the pin punched, apertured non-woven web 118 shown in FIG. 8. Here, the user facing side 20 is highlighted. This view of the pin punched, apertured non-woven web 118 is taken at an angle to the user facing side 20 to highlight details with respect to the plurality of apertures 120 formed therein.

FIG. 9 shows a plurality of the apertures 120, highlighting the star shape imparted therein by the star-shaped pins 80. The distal side 46 (distal area 56) defined by the apertures 120 is discernable in this view. The aperture fidelity AF of the distal sides 46 of the apertures 120 is visually appreciable in this enlarged photograph.

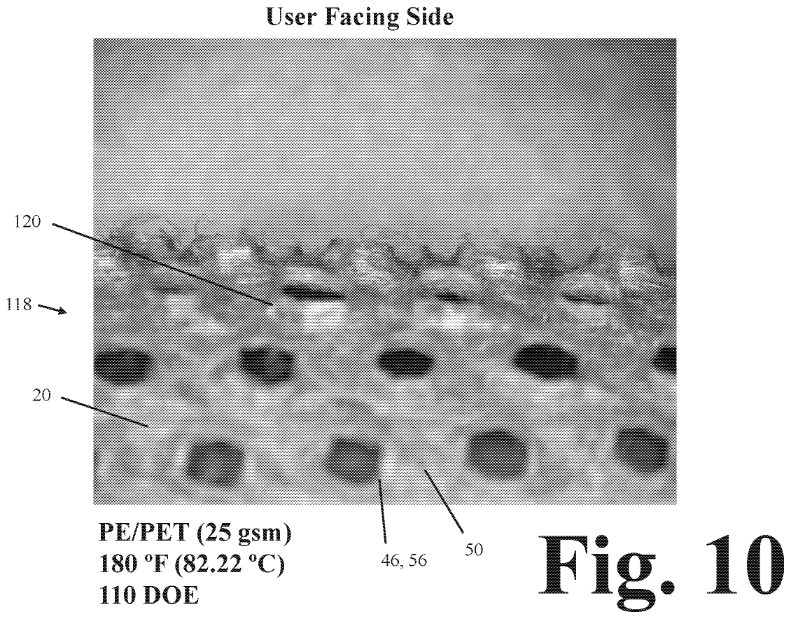

FIG. 10 is a second, enlarged photograph of the pin punched, apertured non-woven web 118 shown in FIGS. 8 and 9 where the photograph is taken at a different angle to the angle of the photograph provided in FIG. 9 to highlight details with respect to the plurality of apertures 120 formed therein by pin punching according to the present invention. The aperture fidelity AF of the distal sides 46 of the apertures 120 also is visually appreciable in this enlarged photograph.

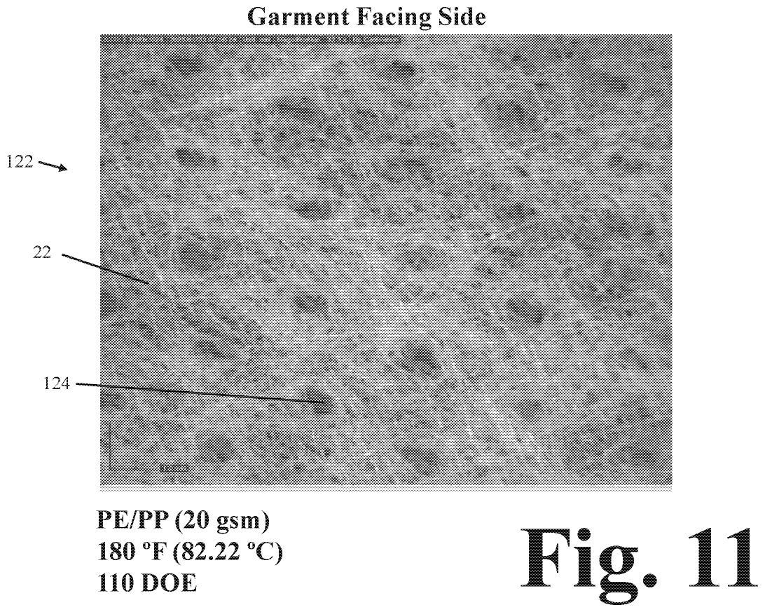

FIG. 11 is an enlarged photograph of another embodiment of a pin punched, apertured non-woven web 122 according to the present invention. Specifically, FIG. 11 shows the garment facing side 22 of the pin punched, apertured non-woven web 122. The pin punched, apertured non-woven web 122 includes a plurality of apertures 124 that have a star shape, having been punched via a plurality of star shaped pins 80 illustrated in FIG. 5.

The pin punched, apertured non-woven web 122 is contemplated to be made from a combination of polyethylene (“PE”) and polypropylene (“PP”). This combination is referred to herein as “PE/PP.” In this figure, the pin punched, apertured non-woven web 122 has a basis weight of 20 grams per square meter (“gsm”).

In the embodiment illustrated in FIG. 11, the PE/PP material employed for the pin pinched, apertured non-woven web 122 is a material referred to as a “Bico” material, meaning that the fibers are bi-component fibers, the details of which are known to those skilled in the art, as noted above. Accordingly, further details concerning the bi-component material are not provided herein.

As is apparent from FIG. 11, the apertures 124 have well-defined shapes that are consistent with the star-shaped pins that result in the formation thereof. As with the pin punched, apertured non-woven web 118, that the apertures 124 have a high aperture fidelity AF within a range of 0.80-1.00, as discussed hereinabove.

To appreciate other aspects of the pin punched, apertured non-woven web 122, the Applicant provides Table 2, below. Table 2 compares selected aspects of the pin punched, apertured non-woven web 122 with the pre-cursor non-woven web prior to pin punching.

TABLE 2

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 20 gsm PE/PP Bico | 278 | 309 | 8.1 | 13.4 | 3.9 |
| Non-woven | | | | | |
| Star 2 Punched 20 | 287 | 319 | 7.4 | 10.2 | 3.6 |
| gsm PE/PP Non- | | | | | |
| woven (180 °F | | | | | |
| (82.22 °C)) | | | | | |
| (110 DOE) | | | | | |

As provided in Table 2, the unapertured, precursor non-woven web 26 had a thickness or loft of 278 μm. After pin punching, the pin punched, apertured non-woven web 122 had an embossed thickness (or loft) of 287 μm. As in the prior embodiment, the thickness/loft 72 of the aperture 42, shown in FIG. 4, is the same parameter as the embossed thickness (or loft) reported in Table 2.

As provided in Table 2, the unapertured, precursor non-woven web 26 had an air permeability of 309 m³/m²/min. After being pin punched, the air permeability of the pin punched, apertured non-woven 122 increased to 319 m³/m²/min.

The unapertured, precursor non-woven web 26 had an open area of 8.1%. After being pin punched, the pin-punched, apertured non-woven web 122 had an open area of 7.4%.

For the embodiment illustrated in FIG. 11, it is noted that the star-shaped pins pierced the unapertured, precursor non-woven web 26 at 180 °F or 82.22 °C. The depth of engagement was 0.110 inches (2.79 mm).

The pin punched, apertured non-woven web 122 was measured as exhibiting a strikethrough of 14.15 seconds. In addition, the pin punched, apertured non-woven web 122 had a rewet of 0.113 grams.

The MD handle of the non-woven precursor web 26 was 13.4 g. The TD handle of the non-woven precursor web 26 was 3.9 g. The MD handle of the pin punched, apertured non-woven web 122 was measured as 10.2 g. The TD handle was measured as 3.6 g.

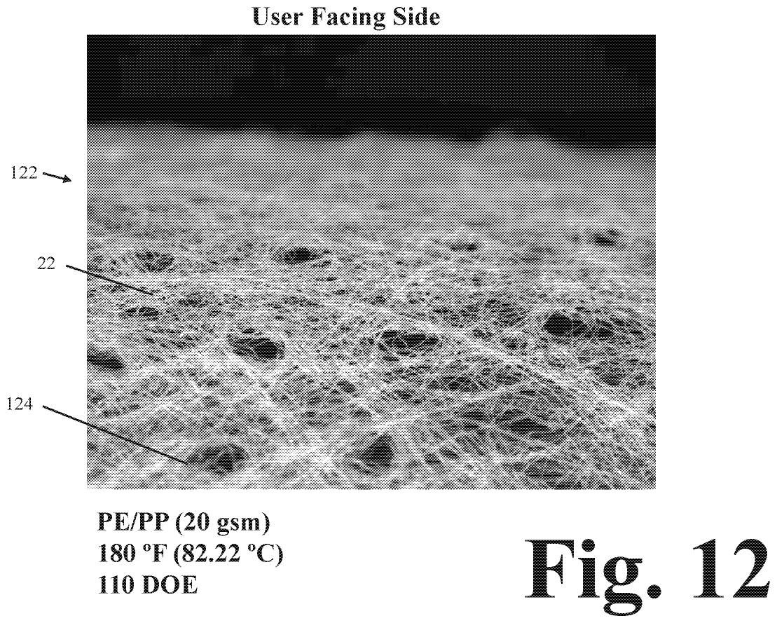

FIG. 12 is an enlarged photograph of the pin punched, apertured non-woven web 122 shown in FIG. 11. Here, the user facing side 20 is highlighted. This view of the pin punched, apertured non-woven web 122 is taken at an angle to the user facing side 20 to highlight details with respect to the plurality of apertures 124 formed therein. As before, the aperture fidelity AF of the distal sides 46 of the apertures 124 is visually appreciable in this enlarged photograph.

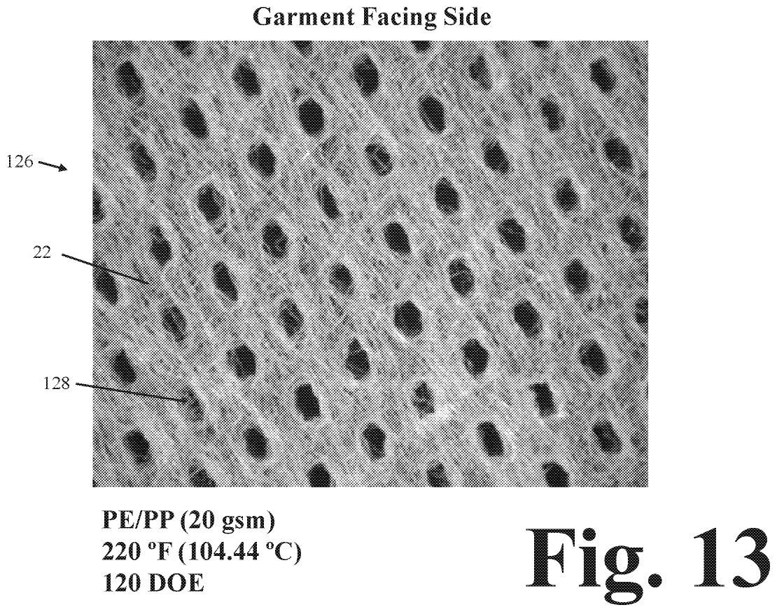

FIG. 13 is an enlarged photograph of another embodiment of a pin punched, apertured non-woven web 126 according to the present invention. Specifically, FIG. 13 shows the garment facing side 22 of the pin punched, apertured non-woven web 126. The pin punched, apertured non-woven web 126 includes a plurality of apertures 128 that have a star shape, having been punched via a plurality of star shaped pins 80 illustrated in FIG. 5.

The pin punched, apertured non-woven web 126 is contemplated to be made from a combination of polyethylene (“PE”) and polypropylene (“PP”). As before, this combination is referred to herein as “PE/PP.” In this figure, the pin punched, apertured non-woven web 126 has a basis weight of 20 grams per square meter (“gsm”).

In the embodiment illustrated in FIG. 13, the PE/PP material employed for the pin pinched, apertured non-woven web 126 is a “Bico” material, meaning that the fibers are bi-component fibers, the details of which are known to those skilled in the art. Accordingly, further details concerning the bi-component material are not provided herein.

As is apparent from FIG. 13, the apertures 128 have well-defined shapes that are consistent with the star-shaped pins that result in the formation thereof. As with prior examples of the pin punched, apertured non-woven web, that the apertures 128 have a high aperture fidelity AF within a range of 0.80-1.00, as discussed hereinabove.

To appreciate other aspects of the pin punched, apertured non-woven web 126, the Applicant provides Table 3, below. Table 3 compares selected aspects of the pin punched, apertured non-woven web 126 with the non-woven pre-cursor web 26 prior to pin punching.

TABLE 3

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 20 gsm PE/PET | 278 | 309 | 8.1 | 13.4 | 3.9 |
| Bico Non-woven | | | | | |
| Star 2 Punched 20 | 394 | 354 | 13.3 | 15.0 | 4.0 |
| gsm PE/PET Non- | | | | | |
| woven (220 °F | | | | | |
| (104.44 °C)) | | | | | |
| (120 DOE) | | | | | |

As provided in Table 3, the unapertured, pre-cursor non-woven web 26 had a thickness or loft of 278 μm. After pin punching, the pin punched, apertured non-woven web 126 had an embossed thickness (or loft) of 394 μm. As in the prior embodiments, the thickness/loft 72 of the aperture 42, shown in FIG. 4, is the same parameter as the embossed thickness (or loft) reported in Table 3.

As provided in Table 3, the unapertured, precursor non-woven web 26 had an air permeability of 309 m³/m²/min. After being pin punched, the air permeability of the pin punched, apertured non-woven 126 increased to 354 m³/m²/min.

The unapertured, precursor non-woven web 26 had an open area of 8.1%. After being pin punched, the pin-punched, apertured non-woven web 126 had an open area of 13.3%.

For the embodiment illustrated in FIG. 13, it is noted that the star-shaped pins pierced the unapertured, precursor non-woven web 26 at 220 °F or 104.44 °C. The depth of engagement was 0.120 inches (3.05 mm).

The pin punched, apertured non-woven web 126 was measured as exhibiting a strikethrough of 6.79 seconds. In addition, the pin punched, apertured non-woven web 126 had a rewet of 0.190 grams.

The MD handle of the non-woven precursor web 26 was 13.4 g. The TD handle of the non-woven precursor web 26 was 3.9 g. The MD handle of the pin punched, apertured non-woven web 126 was measured as 15.0 g. The TD handle for the pin punched, apertured non-woven web 126 was measured as 4.0 g.

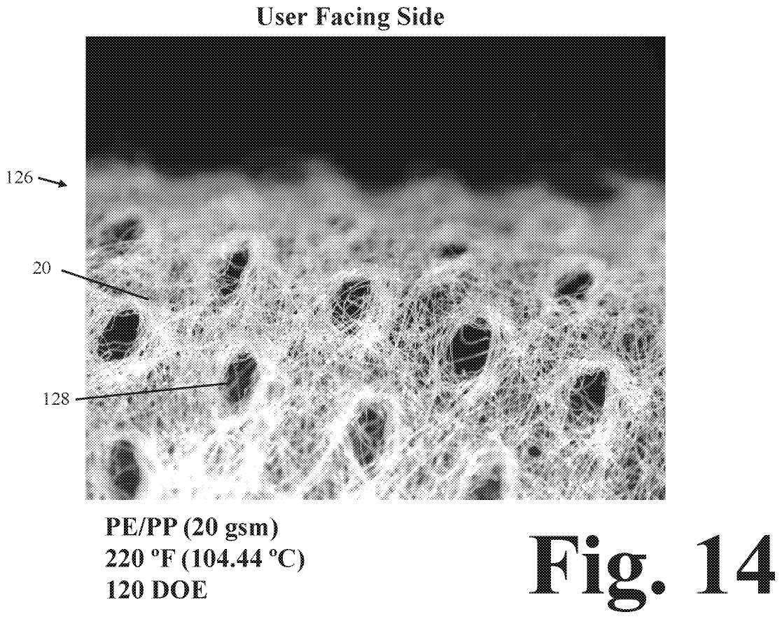

FIG. 14 is an enlarged photograph of the pin punched, apertured non-woven web 126 shown in FIG. 13. Here, the user facing side 20 is highlighted. This view of the pin punched, apertured non-woven web 126 is taken at an angle to the user facing side 20 to highlight details with respect to the plurality of apertures 128 formed therein. As before, the aperture fidelity AF of the distal sides 46 of the apertures 128 is visually appreciable in this enlarged photograph.

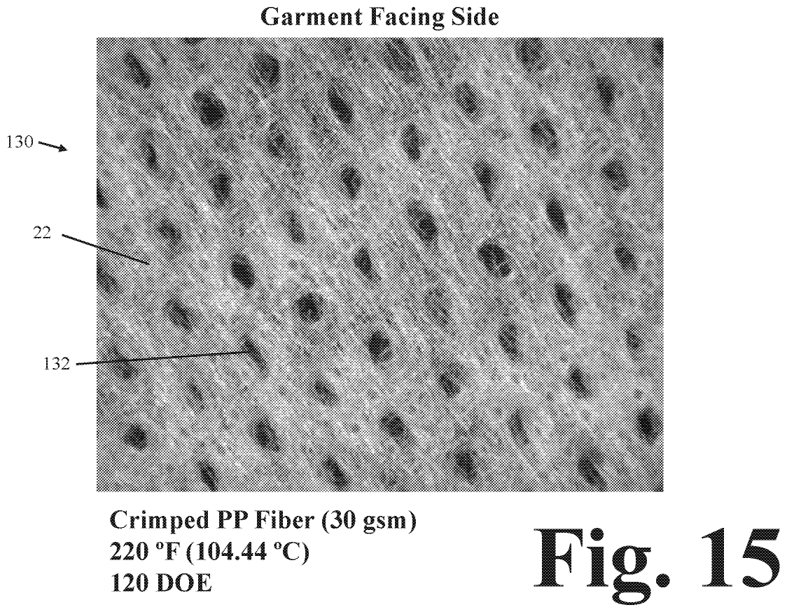

FIG. 15 is an enlarged photograph of another embodiment of a pin punched, apertured non-woven web 130 according to the present invention. Specifically, FIG. 15 shows the garment facing side 22 of the pin punched, apertured non-woven web 130. The pin punched, apertured non-woven web 130 includes a plurality of apertures 132 that have a star shape, having been punched via a plurality of star shaped pins 80 illustrated in FIG. 5.

The pin punched, apertured non-woven web 130 was made from a crimped polypropylene (“PP”) fiber. In this embodiment, the pin punched, apertured non-woven web 130 has a basis weight of 30 grams per square meter (“gsm”).

In the embodiment illustrated in FIG. 15, the crimped PP fiber material employed for the pin pinched, apertured non-woven web 130 has a composition that is known to those skilled in the art. Accordingly, further details concerning the crimped PP fiber material are not provided herein.

As is apparent from FIG. 15, the apertures 132 have well-defined shapes that are consistent with the star-shaped pins that result in the formation thereof. As with prior examples of the pin punched, apertured non-woven web, that the apertures 132 have a high aperture fidelity AF within a range of 0.80-1.00, as discussed hereinabove.

To appreciate other aspects of the pin punched, apertured non-woven web 130, the Applicant provides Table 4, below. Table 4 compares selected aspects of the pin punched, apertured non-woven web 130 with the non-woven pre-cursor web 26 prior to pin punching.

TABLE 4

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 30 gsm PE/PET | 323 | 171 | 3.2 | 17.6 | 5.2 |
| Bico Non-woven | | | | | |
| Star 2 Punched 30 | 567 | 246 | 8.3 | 14.6 | 5.0 |
| gsm PP Crimped | | | | | |
| Non-woven (220 °F | | | | | |
| (104.44 °C)) | | | | | |
| (120 DOE) | | | | | |

As provided in Table 4, the unapertured, pre-cursor non-woven web 26 had a thickness or loft of 323 μm. After pin punching, the pin punched, apertured non-woven web 130 had an embossed thickness (or loft) of 567 μm. As in the prior embodiments, the thickness/loft 72 of the aperture 42, shown in FIG. 4, is the same parameter as the embossed thickness (or loft) reported in Table 4.

As provided in Table 4, the unapertured, precursor non-woven web 26 had an air permeability of 171 m³/m²/min. After being pin punched, the air permeability of the pin punched, apertured non-woven 130 increased to 246 m³/m²/min.

The unapertured, precursor non-woven web 26 had an open area of 3.2%. After being pin punched, the pin-punched, apertured non-woven web 130 had an open area of 8.3%.

For the embodiment illustrated in FIG. 15, it is noted that the star-shaped pins pierced the unapertured, precursor non-woven web 26 at 220 °F or 104.44 °C. The depth of engagement was 0.120 inches (3.05 mm).

The pin punched, apertured non-woven web 130 was measured as exhibiting a strikethrough of 7.79 seconds. In addition, the pin punched, apertured non-woven web 130 had a rewet of 0.098 grams.

The MD handle of the non-woven precursor web 26 was 17.6 g. The TD handle of the non-woven precursor web 26 was 5.2 g. The MD handle of the pin punched, apertured non-woven web 130 was measured as 14.6 g. The TD handle of the pin punched, apertured non-woven web 130 was measured as 5.0 g.

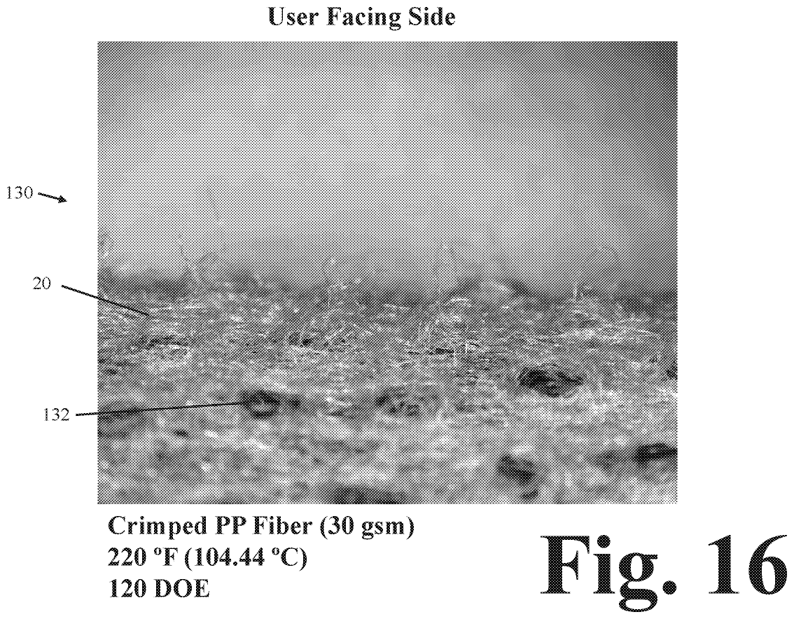

FIG. 16 is an enlarged photograph of the pin punched, apertured non-woven web 130 shown in FIG. 15. Here, the user facing side 20 is highlighted. This view of the pin punched, apertured non-woven web 130 is taken at an angle to the user facing side 20 to highlight details with respect to the plurality of apertures 132 formed therein. As before, the aperture fidelity AF of the distal sides 46 of the apertures 132 is visually appreciable in this enlarged photograph.

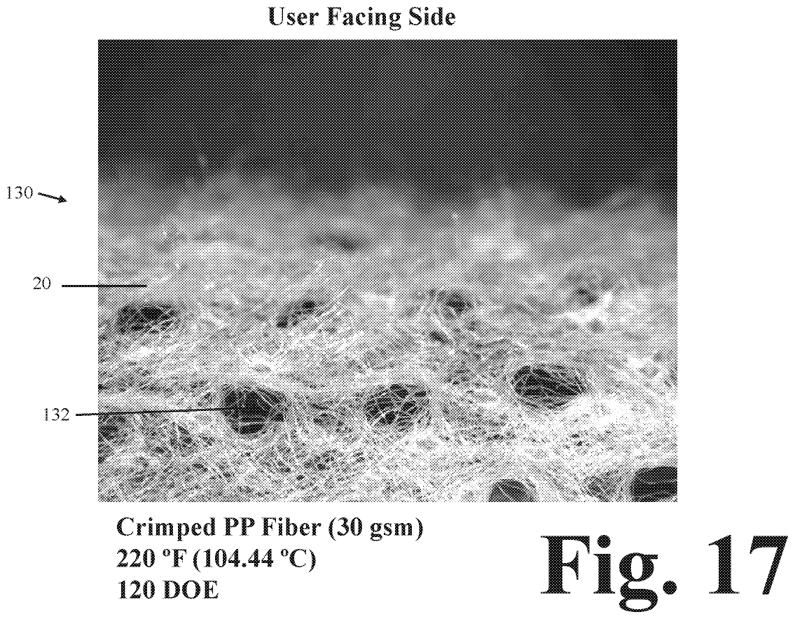

FIG. 17 is an enlarged photograph providing a second view of the user facing side of the pin punched, apertured non-woven web 130 shown in FIGS. 15 and 16. Here again, the aperture fidelity AF is visually apparent.

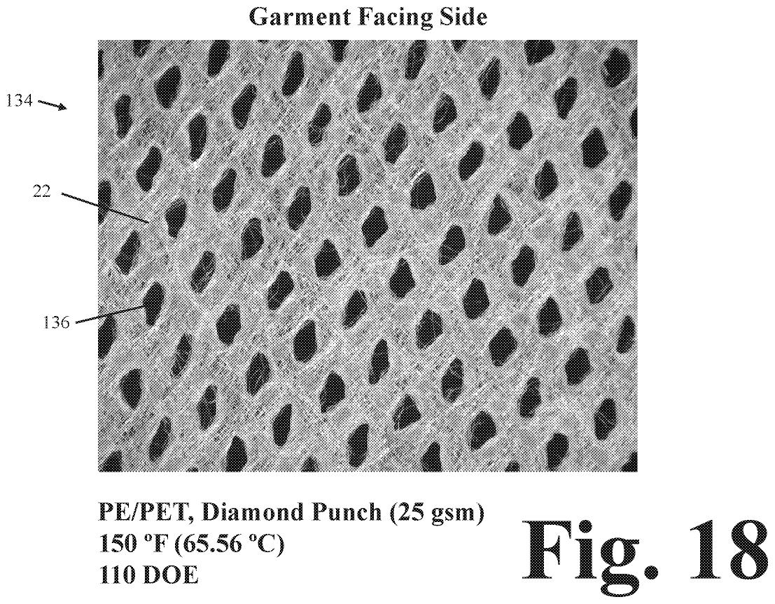

FIG. 18 is an enlarged photograph of another embodiment of a pin punched, apertured non-woven web 134 according to the present invention. Specifically, FIG. 18 shows the garment facing side 22 of the pin punched, apertured non-woven web 134. The pin punched, apertured non-woven web 134 includes a plurality of apertures 136 that have a diamond shape.

The pin punched, apertured non-woven web 134 was made from a non-woven web combining polyethylene (“PE”) and polyethylene terephthalate (“PET”). As noted above, this combination is referred to herein as “PE/PET.” In this embodiment, the pin punched, apertured non-woven web 134 has a basis weight of 25 grams per square meter (“gsm”).

In the embodiment illustrated in FIG. 18, the PE/PET fiber material employed for the pin pinched, apertured non-woven web 134 has a composition that is known to those skilled in the art. Accordingly, further details concerning the PE/PET fiber material are not provided herein.

As is apparent from FIG. 18, the apertures 136 have well-defined shapes that are consistent with the diamond-shaped pins that result in the formation thereof. As with prior examples of the pin punched, apertured non-woven web, that the apertures 134 have a high aperture fidelity AF within a range of 0.80-1.00, as discussed hereinabove.

To appreciate other aspects of the pin punched, apertured non-woven web 134, the Applicant provides Table 5, below. Table 5 compares selected aspects of the pin punched, apertured non-woven web 134 with the non-woven pre-cursor web 26 prior to pin punching.

TABLE 5

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 25 gsm PE/PET | 168 | 141 | 2.9 | 10.1 | 4.3 |
| Non-woven | | | | | |
| Diamond Punched | 524 | 294 | 17.8 | 9.6 | 3.9 |
| 25 gsm PE/PET | | | | | |
| Non-woven (150 °F | | | | | |
| (65.56 °C)) | | | | | |
| (110 DOE) | | | | | |

As provided in Table 5, the unapertured, pre-cursor non-woven web 26 had a thickness or loft of 168 μm. After pin punching, the pin punched, apertured non-woven web 134 had an embossed thickness (or loft) of 524 μm. As in the prior embodiments, the thickness/loft 72 of the aperture 42, shown in FIG. 4, is the same parameter as the embossed thickness (or loft) reported in Table 5.

As provided in Table 5, the unapertured, precursor non-woven web 26 had an air permeability of 141 m³/m²/min. After being pin punched, the air permeability of the pin punched, apertured non-woven 134 increased to 294 m³/m²/min.

The unapertured, precursor non-woven web 26 had an open area of 2.9%. After being pin punched, the pin-punched, apertured non-woven web 134 had an open area of 17.8%.

For the embodiment illustrated in FIG. 18, it is noted that the diamond-shaped pins pierced the unapertured, precursor non-woven web 26 at 150 °F or 65.56 °C. The depth of engagement was 0.110 inches (2.79 mm).

The pin punched, apertured non-woven web 134 was measured as exhibiting a strikethrough of 4.18 seconds. In addition, the pin punched, apertured non-woven web 134 had a rewet of 0.130 grams.

The MD handle of the non-woven precursor web 26 was 10.1 g. The TD handle of the non-woven precursor web 26 was 4.3 g. The MD handle of the pin punched, apertured non-woven web 134 was measured as 9.6 g. The TD handle of the pin punched, apertured non-woven web 134 was measured as 3.9 g.

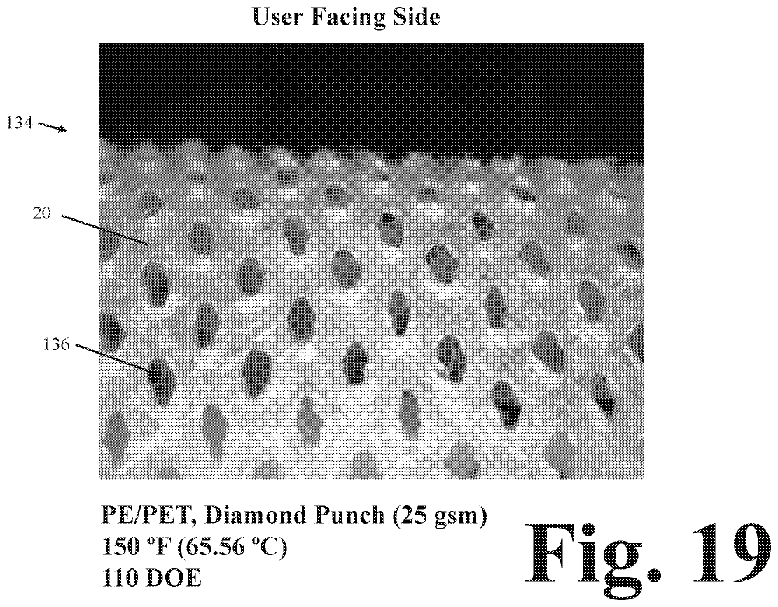

FIG. 19 is an enlarged photograph of the pin punched, apertured non-woven web 134 shown in FIG. 18. Here, the user facing side 20 is highlighted. This view of the pin punched, apertured non-woven web 134 is taken at an angle to the user facing side 20 to highlight details with respect to the plurality of apertures 136 formed therein. As before, the aperture fidelity AF of the distal sides 46 of the apertures 134 is visually appreciable in this enlarged photograph.

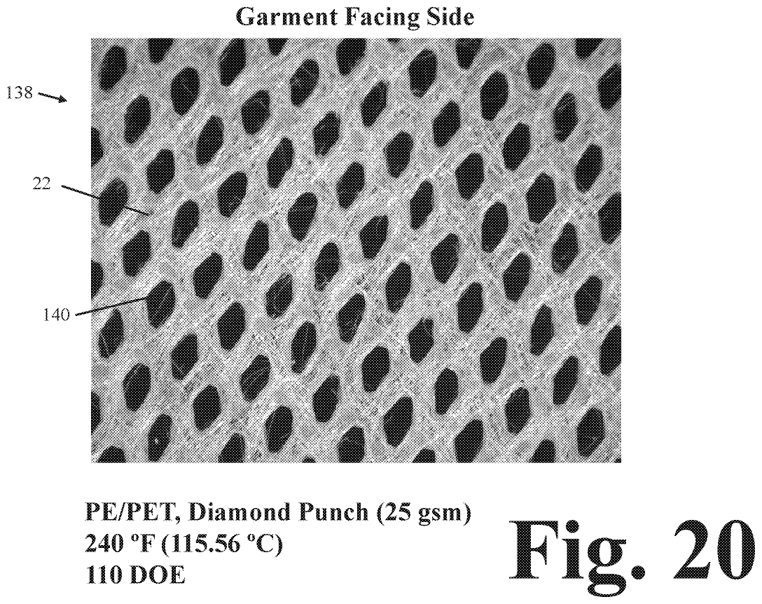

FIG. 20 is an enlarged photograph of another embodiment of a pin punched, apertured non-woven web 138 according to the present invention. Specifically, FIG. 20 shows the garment facing side 22 of the pin punched, apertured non-woven web 138. The pin punched, apertured non-woven web 138 includes a plurality of apertures 140 that have a diamond shape.

The pin punched, apertured non-woven web 138 was made from a non-woven web combining polyethylene (“PE”) and polyethylene terephthalate (“PET”). As noted above, this combination is referred to herein as “PE/PET.” In this embodiment, the pin punched, apertured non-woven web 138 has a basis weight of 25 grams per square meter (“gsm”).

In the embodiment illustrated in FIG. 20, the PE/PET fiber material employed for the pin pinched, apertured non-woven web 138 has a composition that is known to those skilled in the art. Accordingly, further details concerning the PE/PET fiber material are not provided herein.

As is apparent from FIG. 20, the apertures 140 have well-defined shapes that are consistent with the diamond-shaped pins that result in the formation thereof. As with prior examples of the pin punched, apertured non-woven web, that the apertures 140 have a high aperture fidelity AF within a range of 0.80-1.00, as discussed hereinabove.

To appreciate other aspects of the pin punched, apertured non-woven web 138, the Applicant provides Table 6, below. Table 6 compares selected aspects of the pin punched, apertured non-woven web 138 with the non-woven pre-cursor web 26 prior to pin punching.

TABLE 6

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 25 gsm PE/PET | 168 | 141 | 2.9 | 10.1 | 4.3 |
| Non-woven | | | | | |
| Diamond Punched | 493 | 329 | 25.5 | 16.4 | 6.0 |
| 25 gsm PE/PET | | | | | |
| Non-woven (240 °F | | | | | |
| (115.56 °C)) | | | | | |
| (110 DOE) | | | | | |

As provided in Table 6, the unapertured, pre-cursor non-woven web 26 had a thickness or loft of 168 μm. After pin punching, the pin punched, apertured non-woven web 138 had an embossed thickness (or loft) of 493 μm. As in the prior embodiments, the thickness/loft 72 of the aperture 42, shown in FIG. 4, is the same parameter as the embossed thickness (or loft) reported in Table 6.

As provided in Table 6, the unapertured, precursor non-woven web 26 had an air permeability of 141 m³/m²/min. After being pin punched, the air permeability of the pin punched, apertured non-woven 138 increased to 329 m³/m²/min.

The unapertured, precursor non-woven web 26 had an open area of 2.9%. After being pin punched, the pin-punched, apertured non-woven web 138 had an open area of 25.5%.

For the embodiment illustrated in FIG. 20, it is noted that the diamond-shaped pins pierced the unapertured, precursor non-woven web 26 at 240 °F or 115.56 °C. The depth of engagement was 0.110 inches (2.79 mm).

The pin punched, apertured non-woven web 138 was measured as exhibiting a strikethrough of 4.15 seconds. In addition, the pin punched, apertured non-woven web 134 had a rewet of 0.114 grams.

The MD handle of the non-woven precursor web 26 was 10.1 g. The TD handle of the non-woven precursor web 26 was 4.3 g. The MD handle of the pin punched, apertured non-woven web 138 was measured as 16.4 g. The TD handle of the pin punched, apertured non-woven web 138 was measured as 6.0 g.

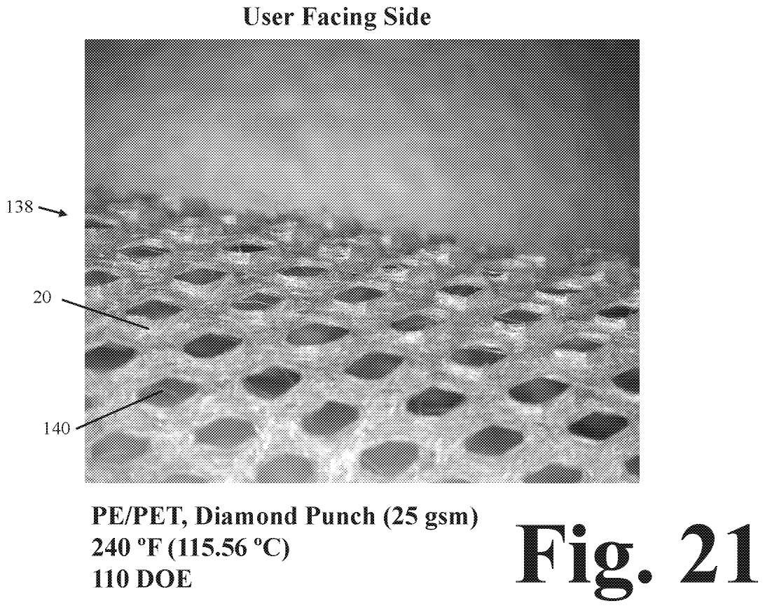

FIG. 21 is an enlarged photograph of the pin punched, apertured non-woven web 138 shown in FIG. 20. Here, the user facing side 20 is highlighted. This view of the pin punched, apertured non-woven web 138 is taken at an angle to the user facing side 20 to highlight details with respect to the plurality of apertures 140 formed therein. As before, the aperture fidelity AF of the distal sides 46 of the apertures 140 is visually appreciable in this enlarged photograph.

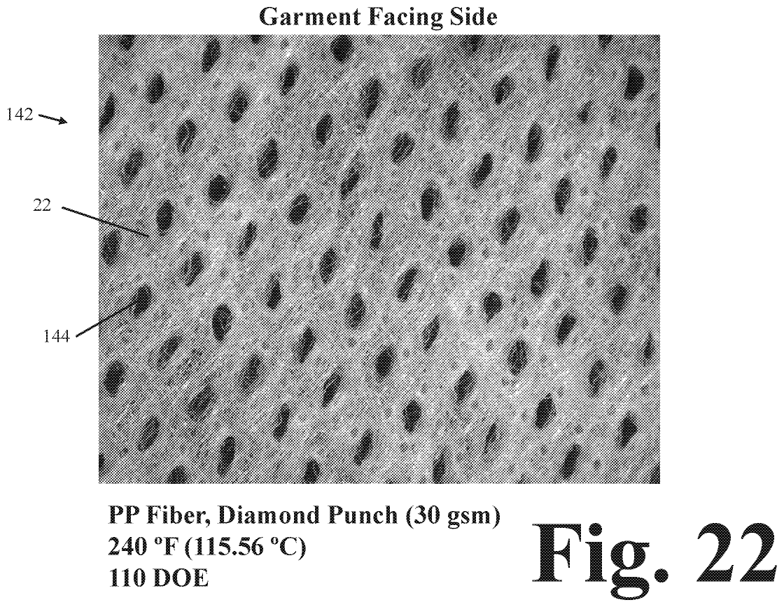

FIG. 22 is an enlarged photograph of another embodiment of a pin punched, apertured non-woven web 142 according to the present invention. Specifically, FIG. 22 shows the garment facing side 22 of the pin punched, apertured non-woven web 142. The pin punched, apertured non-woven web 142 includes a plurality of apertures 144 that have a diamond shape.

The pin punched, apertured non-woven web 142 was made from a non-woven web with fibers made from polypropylene (“PP”). In this embodiment, the pin punched, apertured non-woven web 142 has a basis weight of 30 grams per square meter (“gsm”).

In the embodiment illustrated in FIG. 22, the PP fiber material employed for the pin pinched, apertured non-woven web 142 has a composition that is known to those skilled in the art. Accordingly, further details concerning the PP fiber material are not provided herein.

As is apparent from FIG. 22, the apertures 144 have well-defined shapes that are consistent with the diamond-shaped pins that result in the formation thereof. As with prior examples of the pin punched, apertured non-woven web, that the apertures 144 have a high aperture fidelity AF within a range of 0.80-1.00, as discussed hereinabove.

To appreciate other aspects of the pin punched, apertured non-woven web 138, the Applicant provides Table 7, below. Table 7 compares selected aspects of the pin punched, apertured non-woven web 142 with the non-woven pre-cursor web 26 prior to pin punching.

TABLE 7

| | Embossed | Air | Open | MD | TD |
|---|---|---|---|---|---|
| | Thickness | Permeability | Area | Handle | Handle |
| Description | (μm) | (m³/m²/min) | (%) | (g) | (g) |
| 25 gsm PE/PET | 168 | 141 | 2.9 | 10.1 | 4.3 |
| Non-woven | | | | | |
| Diamond Punched | 593 | 274 | 5.6 | 15.6 | 5.3 |
| 30 gsm PP Non- | | | | | |
| woven (240 °F | | | | | |
| (115.56 °C)) | | | | | |
| (110 DOE) | | | | | |

As provided in Table 7, the unapertured, pre-cursor non-woven web 26 had a thickness or loft of 168 μm. After pin punching, the pin punched, apertured non-woven web 142 had an embossed thickness (or loft) of 593 μm. As in the prior embodiments, the thickness/loft 72 of the aperture 42, shown in FIG. 4, is the same parameter as the embossed thickness (or loft) reported in Table 7.

As provided in Table 7, the unapertured, precursor non-woven web 26 had an air permeability of 141 m³/m²/min. After being pin punched, the air permeability of the pin punched, apertured non-woven 142 increased to 274 m³/m²/min.

The unapertured, precursor non-woven web 26 had an open area of 2.9%. After being pin punched, the pin-punched, apertured non-woven web 142 had an open area of 5.6%.

For the embodiment illustrated in FIG. 22, it is noted that the diamond-shaped pins pierced the unapertured, precursor non-woven web 26 at 240 °F or 115.56 °C. The depth of engagement was 0.110 inches (2.79 mm).

The pin punched, apertured non-woven web 142 was measured as exhibiting a strikethrough of 7.78 seconds. In addition, the pin punched, apertured non-woven web 142 had a rewet of 0.085 grams.

The MD handle of the non-woven precursor web 26 was 10.1 g. The TD handle of the non-woven precursor web 26 was 4.3 g. The MD handle of the pin punched, apertured non-woven web 142 was measured as 15.4 g. The TD handle of the pin punched, apertured non-woven web 142 was measured as 5.3 g.

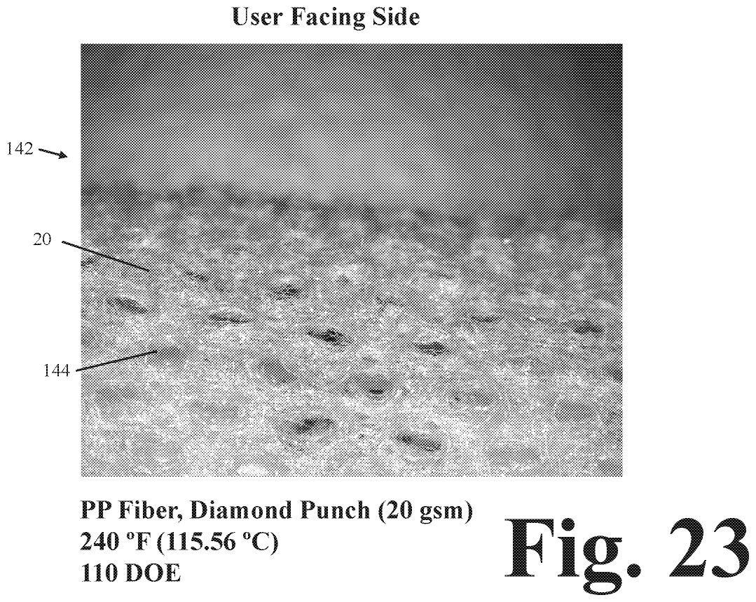

FIG. 23 is an enlarged photograph of the pin punched, apertured non-woven web 142 shown in FIG. 22. Here, the user facing side 20 is highlighted. This view of the pin punched, apertured non-woven web 142 is taken at an angle to the user facing side 20 to highlight details with respect to the plurality of apertures 144 formed therein. As before, the aperture fidelity AF of the distal sides 46 of the apertures 144 is visually appreciable in this enlarged photograph.

Table 8, provided below, compares various embodiments of a diamond punched non-woven web, illustrating how various temperatures have an impact on the properties of the non-woven web.

TABLE 8

| | Embossed | Air | Open | MD | TD | | |
|---|---|---|---|---|---|---|---|
| | Thickness | Permeability | Area | Handle | Handle | Strikethrough | Rewet |
| Description | (μm) | (m³/m²/min) | (%) | (g) | (g) | (s) | (g) |
| 25 gsm PE/PET | 168 | 141 | 2.9 | 10.1 | 4.3 | — | — |
| Non-woven | | | | | | | |
| Diamond Punched | 524 | 294 | 17.8 | 9.6 | 3.9 | 4.18 | 0.130 |
| 25 gsm PE/PET | | | | | | | |
| Non-woven (150 °F | | | | | | | |
| (65.56 °C)) | | | | | | | |
| (110 DOE) | | | | | | | |
| Diamond Punched | 544 | 323 | 21.3 | 12.7 | 5.3 | 5.10 | 0.142 |
| 25 gsm PE/PET | | | | | | | |
| Non-woven (180 °F | | | | | | | |
| (82.22 °C)) | | | | | | | |
| (110 DOE) | | | | | | | |
| Diamond Punched | 483 | 326 | 24.0 | 15.6 | 5.8 | 3.55 | 0.089 |
| 25 gsm PE/PET | | | | | | | |
| Non-woven (220 °F | | | | | | | |
| (104.44 °C)) | | | | | | | |
| (110 DOE) | | | | | | | |
| Diamond Punched | 493 | 329 | 25.5 | 16.4 | 6.0 | 4.15 | 0.114 |
| 25 gsm PE/PET | | | | | | | |
| Non-woven (240 °F | | | | | | | |
| (115.56 °C)) | | | | | | | |
| (110 DOE) | | | | | | | |

Separately, data was acquired for a non-woven web that was made from a material combining polyethylene (“PE”) and polylactic acid (“PLA”). This material is referred to as a “PE/PLA” material herein.

Table 9, provided below, provides data associated with a PE/PLA non-woven web that was pierced by diamond-shaped pins with a penetration of 0.110 inches (2.94 mm) (DOE 110).

TABLE 9

| | Embossed | Air | Open | MD | TD | | |
|---|---|---|---|---|---|---|---|
| | Thickness | Permeability | Area | Handle | Handle | Strikethrough | Rewet |
| Description | (μm) | (m³/m²/min) | (%) | (g) | (g) | (s) | (g) |
| 18 gsm PE/PLA | 146 | 233 | 15.1 | 5.6 | 2.4 | — | — |
| Non-woven | | | | | | | |
| Diamond Punched | 450 | 390 | 19.6 | 5.5 | 2.6 | 4.33 | 0.235 |
| 18 gsm PE/PLA | | | | | | | |
| Non-woven (150 °F | | | | | | | |
| (65.56 °C)) | | | | | | | |
| (110 DOE) | | | | | | | |
| Diamond Punched | 465 | 393 | 21.0 | 6.5 | 2.8 | 3.98 | 0.431 |
| 18 gsm PE/PLA | | | | | | | |
| Non-woven (180 °F | | | | | | | |
| (82.22 °C)) | | | | | | | |
| (110 DOE) | | | | | | | |
| Diamond Punched | 450 | 421 | 25.3 | 7.2 | 2.6 | 3.59 | 0.205 |
| 25 gsm PE/PLA | | | | | | | |
| Non-woven (220 °F | | | | | | | |
| (104.44 °C)) | | | | | | | |
| (110 DOE) | | | | | | | |
| Diamond Punched | 479 | 405 | 23.2 | 7.1 | 2.9 | 3.04 | 0.375 |
| 25 gsm PE/PLA | | | | | | | |
| Non-woven (240 °F | | | | | | | |
| (115.56 °C)) | | | | | | | |
| (110 DOE) | | | | | | | |

It is noted that the invention, as discussed above, primarily addresses the orientation where the apertures 42 point toward the user. Those skilled in the art also refer to this orientation as a “male side up” orientation. As is apparent from FIG. 4, for example, this means that the distal 46 of the aperture 42 is directed toward the user.

It is noted that the orientation for the apertures 42 that is illustrated in FIG. 4 is contemplated to be the orientation employed when the pin punched, apertured non-woven web 40 is employed as the ADL layer 14.

It is contemplated, however, that the pin punched, apertured non-woven web 40 of the present invention may be used in the orientation where the apertures 42 point toward the garment. In other words, the apertures 42 may extend from the garment facing side of the web. This is opposite to the orientation illustrated in FIG. 4. This is also referred to as the “female side up” orientation.

The female side up orientation is contemplated to be employed when the pin punched, apertured non-woven web 40 is used as the topsheet 12. With the female side up, it is contemplated that the pin punched, apertured non-woven web 40 will present a softer feel to the user. For this reason, the female side up orientation is contemplated to be preferred when the pin punched, apertured non-woven web 40 is employed as the topsheet 12.

Still further, the pin punched, apertured non-woven web 40 may be manufactured to have a different phobicity when as a topsheet 12 as opposed to being employed as an ADL layer 14. “Phobicity” refers to the water phobic properties of the material.

When the pin punched, apertured non-woven web 40 is employed as a topsheet 12, it is contemplated that the pin punched, apertured non-woven web 40 may be manufactured to have a higher phobicity. A higher phobicity is contemplated to result in a material that has a drier feel to the user.

On the other hand, when the pin punched, apertured non-woven web 40 is employed as the ADL layer 14 and is separated from the user by the topsheet 12, a lower phobicity is desirable. A lower phobicity translates into a higher wettability. A higher wettability translates to improved transmissibility of fluids through the pin punched, apertured non-woven web 40, thereby improving the performance of the pin punched, apertured non-woven web 40.

While certain combinations of materials are described hereinabove for the fibers making up the non-woven web, it is noted that the present invention is not intended to be limited solely to the materials listed. Other materials may be employed without departing from the scope of the present invention. For example, a PET/COPET bico material may be employed. Alternatively, the material may combine polyethylene terephthalate and co-polyethylene terephthalate. Still further polyethylene terephthalate and/or polylactic acid (“PLA”) may be used.

One observation concerning the present invention is that the change in loft from the non-woven precursor web 26 to the pin punched, apertured non-woven web 76 of the present invention lies within a range of 180%-230%. In other words, the pin punched, apertured non-woven web of the present invention has a loft that is 1.8-2.3 time greater than the initial loft of the non-woven precursor web 26. Other ranges for the change in loft are inclusive of any end points from 180% to 230%, including, but not limited to: 180%, 181%, 182%, 183%, 184%, 185%, 186%, 187%, 188%, 189%, 190%, 191%, 192%, 193%, 194%, 195%, 196%, 197% 198%, 199%, 200%, 201%, 202%, 203%, 204%, 205, %, 206%, 207%, 208%, 209%, 210%, 211%, 212%, 213%, 214%, 215%, 216%, 217%, 218%, 219%, 220%, 221%, 222%, 223%, 224%, 225%, 226%, 227%, 228%, 229%, and 230%. In this context, for example, one contemplated range for the change in loft is 205%-211% within the scope of the present invention.

It is contemplated that an average loft of the pin punched, apertured non-woven web 76 will be an average of 200% greater than the loft of the non-woven precursor web 26. In particular, one range for the change in loft lies within a range of 190%-210%. Another contemplated range is 195%-205%.

Concerning the change in open area from the non-woven precursor web 26 to the pin punched, apertured non-woven web 76 of the present invention, it is contemplated that the open area increases from 1.5-9.0 times. In other words, the open area of the pin punched, apertured non-woven web of the present invention is 1.5-9.0 times greater than the open area of the non-woven precursor web 26. Other ranges that also are contemplated to fall within the scope of the present invention are defined by any two of the following end points, inclusive of the endpoints: 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, and 9.0. One contemplated range includes a change in loft of 3.1-6.2, for example.

Concerning the open area, it is contemplated that open area of the pin punched, apertured non-woven web 76 of the present invention to the open area of the non-woven precursor web 26 will be about 4.5. As such, one range for this ratio falls within a range of 4.0-5.0, 4.3-4.7, and 4.4-4.6.

Concerning the temperature of the pins 34, 80, is it noted that a contemplated range of temperatures is 150-240 °F (65.56-115.56 °C). While not limiting the invention, specific temperatures include 150 °F (65.56 °C), 180 °F (82.22 °C), 220 °F (104.44 °C), and 240 °F (115.56 °C). It is contemplated that the temperature of the pins 34, 80 will exceed the softening point of the material(s) forming the fibers but not exceed the melting point of the material(s) forming the fibers. This is contemplated that a temperature satisfying these parameters will produce a defining edge 84 that satisfies suitable conditions for aperture fidelity AF as discussed above.

Still further, one aspect of the materials employed to create the pin punched, apertured non-woven web 40 of the present invention is associated with the glass transition temperature (“GTT”) of the material forming the fibers 28 of the pin punched, apertured non-woven web 40. Specifically, the GTT of the composition forming the fibers 28 is contemplated to be of interest to the formation of the pin punched, apertured non-woven web 40 of the present invention.

While not intending to limit the present invention, it is noted that, when the GTT of the material or combination of materials forming the fibers 28 is lower, the apertures 42 in the pin punched, apertured non-woven web 40 have a more easily identified shape that is consistent with the pins forming the apertures 42. In other words, when the GTT is lower, the apertures 42 in the pin punched, apertured non-woven web 40 tend to have higher aperture fidelity AF.

In connection with the pin punched, apertured non-woven web 40 of the present invention, a “low” GTT is defined as a temperature between 5° and 85 °C (122-185 °F). One specific range for the GTT includes, but are not limited to, 67-81 °C (152.6-177.8 °F). Another contemplated range is 55-65 °C (131-149 °F).

For purposes of the present invention, ranges for the GTT may be defined by any whole number temperatures between 50-85 °C, inclusive of the endpoints. This includes, but is not limited to: 50 °C, 51 °C, 52 °C, 53 °C, 54 °C, 55 °C, 56 °C, 57 °C, 58 °C, 59 °C, 60 °C, 61 °C, 62 °C, 63 °C, 64 °C, 65 °C, 66 °C, 67 °C, 68 °C, 69 °C, 70 °C, 71 °C, 72 °C, 73 °C, 74 °C, 75 °C, 76 °C, 77 °C, 78 °C, 79 °C, 80 °C, 81 °C, 82 °C, 83 °C, 84 °C, and 85 °C. Accordingly, the GTT may lie within a range of 52-79 °C in one contemplated embodiment. In another, the GTT may lie in a range of 64-71 °C, for example.

From the foregoing, the definition of a low glass transition temperature (low GTT) material means a material with a GTT between 50-85 °C.

Concerning the materials that may be employed to construct the fibers 28, any of the materials discussed herein may be employed either alone or together with other materials also described herein. Still further, although not discussed, the present invention is contemplated to encompass any singular material or combination of materials with a low GTT between 50-85 °C. It is noted that the number of materials that may be employed to construct the fibers may include one, two, three, or more materials without departing from the scope of the present invention.

Concerning the fibers 28, the present invention is contemplated to be applicable to a pin punched, apertured non-woven web 40 having fibers 28 of any suitable weight (also referred to as a “size”), as should be apparent to those skilled in the art. Among the fibers 28 contemplated to be employed, the present invention broadly encompasses fibers 28 with a weight from 1 to 10 Den (Deniers).

As should be apparent to those skilled in the art, the term “Denier” is a unit of measurement used to determine the thickness of individual fibers in textiles and fabrics. The term refers to the mass of the material in grams per 9,000 meters of fiber. A higher denier count indicates a thicker and more durable fabric, while a lower denier count indicates a finer and lighter fabric. For example, a denier count of 1 means that 9,000 meters of the fiber weighs one gram.

As noted above, the present invention is contemplated to encompass, but not be limited to, fibers 28 with a weight from 1 to 10 Den, inclusively. This includes any range of fiber 28 weights from 1 to 10 Den as defined by, and inclusive of, any of the following endpoints: 1 Den, 2 Den, 3 Den, 4 Den, 5 Den, 6 Den, 7 Den, 8 Den, 9 Den, and 10 Den. One contemplated, non-limiting range for the size/weight of the fibers 28 is 1-9 Den, for example.

The present invention also is intended to encompass pin punched, apertured non-woven webs 40 that include one or more treatments. For example, the pin punched, apertured non-woven web 40 may be treated with a hydrophobic material, such as a spray or coating. Still further, the pin punched, apertured non-woven web 40 may incorporate a surfactant. Other treatments and coating may be applied without departing from the scope of the present invention.

The foregoing discussion has focused on pin-punched, apertured non-woven webs 40, 76, 118, 122, 126, 130, 134, 138, 142 that are constructed from a single non-woven precursor web 26. However, the present invention is not limited solely to pin-punched, apertured non-woven webs 40, 76, 118, 122, 126, 130, 134, 138, 142 that are constructed from a single non-woven precursor web 26. To the contrary, the present invention also encompasses a pin-punched, apertured non-woven web that combines two non-woven webs together. The present invention also encompasses an article that incorporates a double-layered pin-punched, apertured non-woven web.

The present invention also encompasses pin-punched, apertured non-woven webs that combines more than two non-woven webs together. The present invention also encompasses an article that incorporates a multi-layered pin-punched, apertured non-woven web.

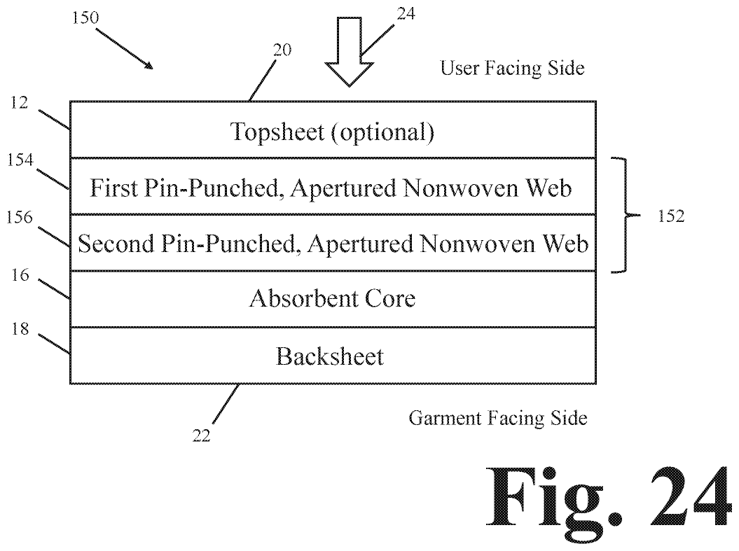

FIG. 24 illustrates an absorbent product 150 similar to the absorbent product 10 illustrated in FIG. 1.

The absorbent product 150 illustrated in FIG. 24 differs from the absorbent product 10 in that the ADL 14 has been replaced by an ADL 152. Here, the ADL 152 comprises a first pin-punched, apertured non-woven web 154 and a second pin-punched, apertured non-woven web 156 that are connected together, as discussed in greater detail hereinbelow.

Many of the features and aspects of the absorbent product 150 and the ADL 152 are discussed hereinabove. Accordingly, those details are not repeated here. Those features and aspects may be incorporated into and/or exhibited by the absorbent product 150 and ADL 152 as should be apparent to those skilled in the art.

The first pin-punched, apertured non-woven web 154 is contemplated to be made from a material that combined polyethylene and polyethylene terephthalate (“PE/PET”). Similarly, the second pin-punched, apertured non-woven web 156 also is contemplated to be made from a PE/PET material. Since the details of PE/PET materials are discussed hereinabove and are known to those skilled in the art, further details are not provided herein.

It is noted that PE/PET materials are understood by those skilled in the art to be hydrophobic, meaning that the PE/PET material tends to repel water therefrom. It is also known that PE/PET materials may be modified to be hydrophilic, meaning that the material attracts water thereto. One way to transform a PE/PET material from a hydrophobic material into a hydrophilic material is to add a surfactant, as should be apparent to those skilled in the art.

With continued reference to FIG. 24, it is noted that the first pin-punched, apertured non-woven web 154 is contemplated to be hydrophobic. As such, the first pin-punched, apertured non-woven web 154 is contemplated to made from a PE/PET material without further treatment(s). The second pin-punched, apertured non-woven web 156 is contemplated to be hydrophilic. In particular, the second pin-punched, apertured non-woven web 156 is contemplated to be made from a PE/PET material that has been treated with a surfactant to imbue hydrophilic properties therein.

With continued reference to FIG. 24, it should be understood that the present invention is not intended to be limited solely to a construction for an ADL 152 that includes a hydrophobic first pin-punched, apertured non-woven web 154 made from PE/PET and a hydrophilic second pin-punched, apertured non-woven web 156 made from PE/PET. To the contrary, there are multiple variations that are contemplated to fall within the scope of the present invention.

For example, while PE/PET is contemplated for the first pin-punched, apertured non-woven web 154 and the second pin-punched, apertured non-woven web 156, other materials may be employed for either non-woven web, as discussed in connection with FIGS. 1-23 above. Moreover, the first pin-punched, apertured non-woven web 154 need not be made from the same material as the second pin-punched, apertured non-woven web 156.

Second, while the absorbent article 150 is described as placing the hydrophobic first pin-punched, apertured non-woven web 154 atop the hydrophilic second pin-punched, apertured non-woven web 156, other variations also are contemplated to fall within the scope of the present invention.

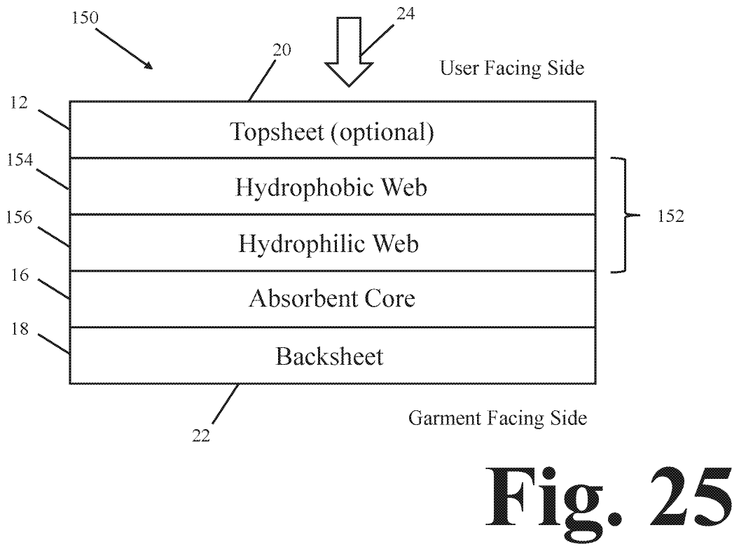

To simplify the discussion that follows, the first pin-punched, apertured non-woven web 154 will be described as a hydrophobic web 154 and the second pin-punched, apertured non-woven web 156 will be described as a hydrophilic web 156. As noted in the embodiment illustrated in FIG. 24, the hydrophobic web 154 is closer to the user facing side 20 of the absorbent article 150. The hydrophilic web 156 is closer to the garment facing side 22. This simplified construction is illustrated in FIG. 25.

Here the hydrophobic web 154 is positioned between the topsheet 12 and the hydrophilic web 156. The hydrophilic web 156, in turn, is positioned between the hydrophobic web 154 and the absorbent core 16.

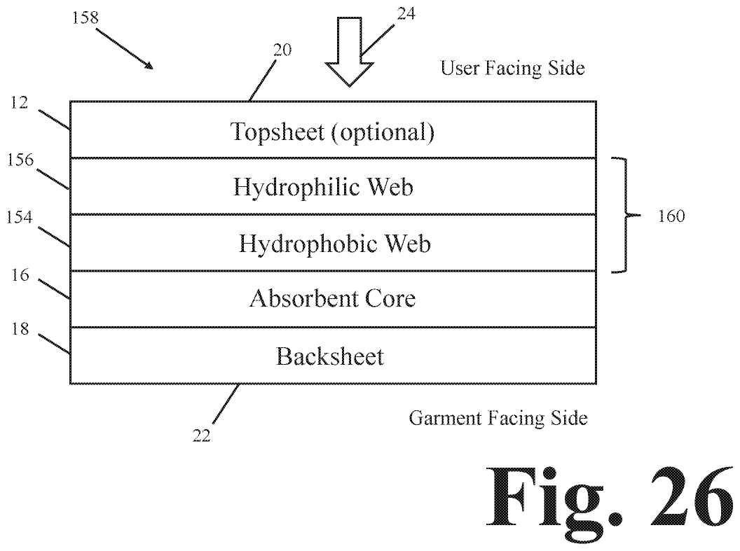

It is noted that FIG. 25 is the same as FIG. 24, except that the appellation for the first pin-punched, apertured non-woven web 154 has been changed to “hydrophobic web” 154 and the appellation for the second pin-punched, apertured non-woven web 156 has been changed to “hydrophilic web” 156. This change is made to facilitate the discussion of FIGS. 26-28.

FIG. 26 is a graphical, cross-sectional representation of an absorbent article 158 where the construction of the ADL 160 differs from the construction of the ADL 152 shown in FIGS. 24 and 25. For the absorbent article 158, the hydrophilic web 156 has been moved to a position closer to the user facing side 20 and the hydrophobic web 154 has been moved closer to the garment facing side 22. In other words, the positions of the hydrophobic web 154 and the hydrophilic web 156 have been reversed by comparison with the absorbent article 150 illustrated in FIG. 24 and in FIG. 25.

As shown in FIG. 26, the hydrophilic web 156 is positioned between the topsheet 12 and the hydrophobic web 154. The hydrophobic web 154, in turn, is positioned between the hydrophilic web 156 and the absorbent core 16.

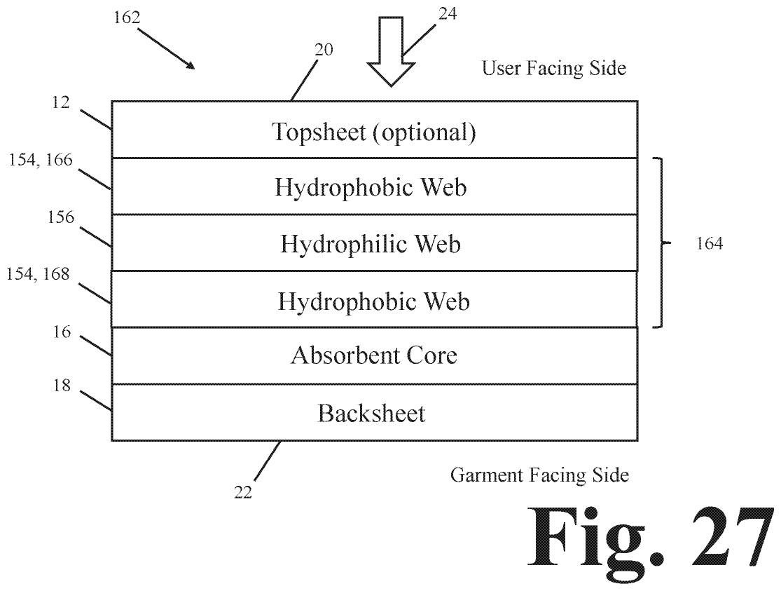

FIG. 27 is a graphical, cross-sectional representation of an absorbent article 162 having an ADL 164.

In this embodiment, the ADL 164 comprises three layers instead of two. Here, the ADL 164 includes two hydrophobic webs 154 sandwiching a hydrophilic web 156 therebetween. Specifically, a first hydrophobic web 166 is disposed adjacent to the topsheet 12. The hydrophilic web 154 is disposed below and adjacent to the first hydrophobic web 166. A second hydrophibic web 168 is positioned beneath the hydrophilic web 154, between the hydrophilic web 154 and the absorbent core 16.

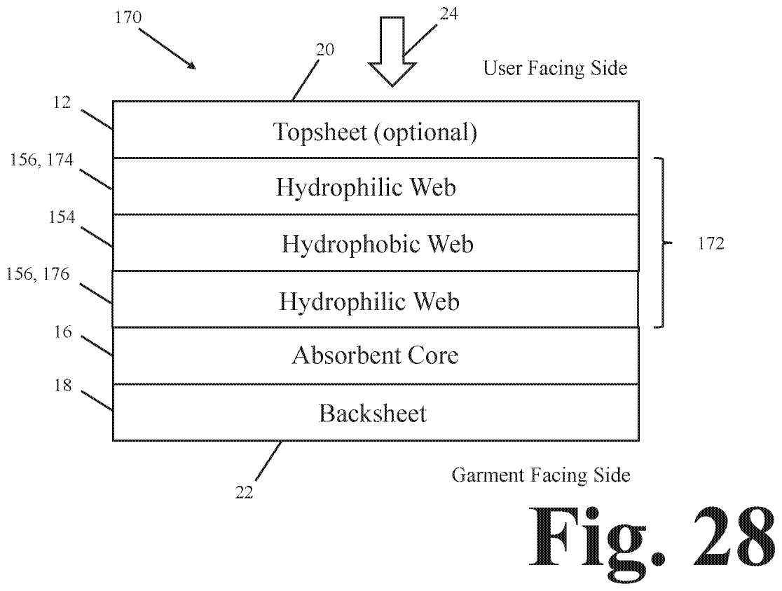

FIG. 28 is a graphical, cross-sectional representation of another absorbent article 170 according to the present invention.

The absorbent article 170 is similar to the absorbent article 162 in FIG. 27 in that the absorbent article 170 includes an ADL 172 made up of three pin-punched, apertured non-woven webs. In this embodiment, the ADL 172 includes two hydrophilic webs 156 that sandwich a hydrophobic web 156.

As illustrated, a first hydrophilic web 174 is disposed adjacent to the topsheet 12, between the topsheet 12 and the hydrophobic web 154. A second hydrophilic web 176 is positioned beneath the hydrophobic web 154, between the hydrophobic web 154 and the absorbent core 16.

It is noted that the three layer ADLs 164, 172 are merely examples of ADLs that are contemplated to fall within the scope of the present invention. It is contemplated that an ADL may be created with four or more pin-punched, apertured non-woven webs.

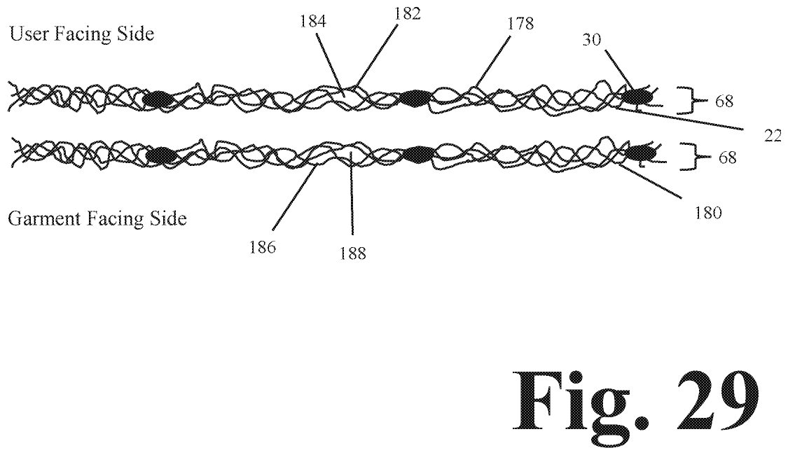

FIGS. 29-31 illustrate the creation of the ADL 152. The discussion of the formation of the ADL 152 may be applied to the other ADLs and variants thereof that are discussed herein, as should be apparent to those skilled in the art.

FIG. 29 illustrates a first precursor non-woven web 178 and a second precursor web 180.

In this illustrated embodiment, the first precursor web 178 and the second precursor web 180 are contemplated to be made from a material that combines polyethylene and polyethylene terephthalate (“PE/PET”), details of which are provided hereinabove. In the illustrated embodiment, the first precursor web 178 is contemplated to embody hydrophobic (water-repelling) properties while the second precursor web 180 is contemplated to embody hydrophilic (water-attracting) properties. The second precursor web 180 is contemplated to be treated with a surfactant to imbue hydrophilic properties thereon.

The first precursor web 178 is contemplated to include first fibers 182 with first voids 184 therebetween. Similarly, the second precursor web 180 is contemplated to be made from second fibers 186 with second voids 188 therebetween. As noted above, the first precursor web 178 and the second precursor web 180 may include optional bond points 30.

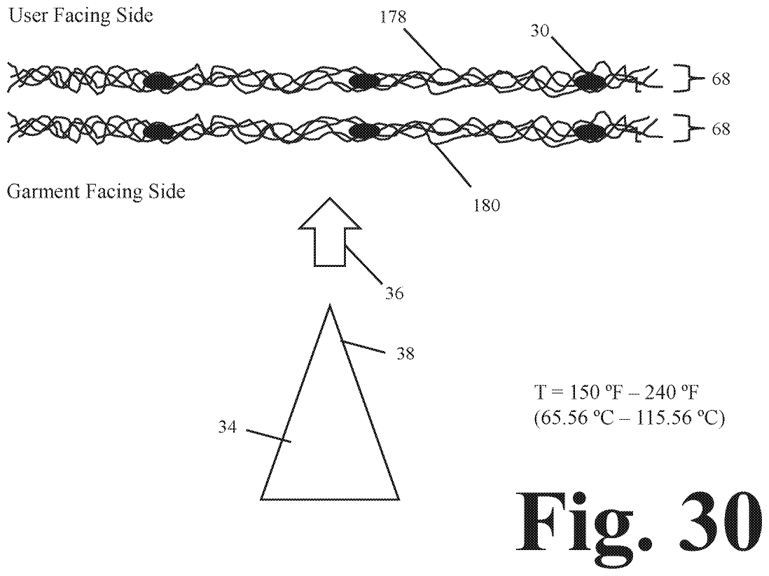

FIG. 30 is a graphical illustration of the formation of the ADL 152 using a pin 34. The pin 34 is pressed through both the first precursor web 178 and the second precursor web 180. Without being limited to theory, it is understood that the heated pin 34 causes some of the first fibers 182 and some of the second fibers 184 to soften and/or melt. The first and second fibers 182, 184 then adhere to one another to form a unified non-woven web that is the ADL 152.

In FIG. 30, the first precursor web 178 and the second precursor web 180 are illustrated as being separate from one another. This is merely for illustration purposes. When the pins 34 are pressed through the two webs 178, 180, it is contemplated that the two webs 178, 180 will be in physical contact with one another.

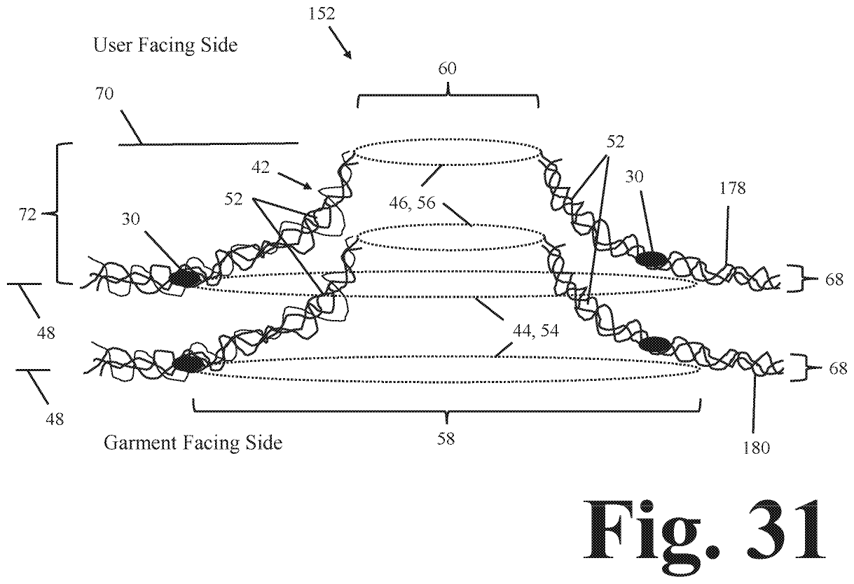

FIG. 31 illustrates the ADL 152 after the pins 34 have created the apertured non-woven material. Here again, the first and second webs 178, 180 are shown as being separated from one another. However, this is merely for illustration purposes. The first and second webs 178, 180 are contemplated to be in physical contact with one another after being connected to one another via the pins 34.

For purposes of the ADL 152, 160, 164, 172, the combination of hydrophobic webs 154 and hydrophilic webs 156 offers advantages concerning passthrough of the insult and rewet. Without being bound by theory, it is believed that the hydrophilic properties of the hydrophilic web 156 contributes to a pass through of the insult through the ADL 152. Still further, it is believed that the hydrophobic properties of the hydrophobic web 154 contributes to a reduced rewet once the insult has passed through the ADL 152.

In the various illustrations of the ADLs 152, 160, 164, 172, it is contemplated that the first and second webs 178, 180 have the same loft 68. However, the lofts 68 of the first and second webs 178, 180 may differ without departing from the scope of the present invention.

Still further, while a surfactant is contemplated to imbue hydrophilic properties to the hydrophilic web, the present invention is not limited solely to the use of surfactants. Other treatments may be employed without departing from the scope of the present invention.

As should be apparent from the foregoing, the various embodiments of the present invention are intended to encompass, but not be limited to, non-woven webs made from polypropylene (“PP”), polyethylene (“PE”), polyethylene terephthalate (“PET”), polylactic acid (“PLA”), and combinations of these materials, among others as should be apparent to those skilled in the art.

In addition, for those embodiments that combine two or more non-woven webs, those webs may include one or more hydrophilic webs and one or more hydrophobic webs in any particular order. For example, a pin punched, apertured non-woven web is contemplated to combine two hydrophobic webs in touching proximity adjacent to a hydrophobic web. Similarly, another embodiment of the pin punched, apertured non-woven web is contemplated to combine a first hydrophobic web, a second hydrophobic web, and a hydrophilic web, assembled in that order. Other variations also are possible, as should be apparent to those skilled in the art.

As discussed hereinabove, the embodiments of the present invention are exemplary only and are not intended to limit the present invention. Features from one embodiment are interchangeable with other embodiments, as should be apparent to those skilled in the art. As such, variations and equivalents of the embodiments described herein are intended to fall within the scope of the claims appended hereto.

What is claimed is:

1. A pin punched apertured non-woven web, comprising:
a plurality of fibers defining a non-woven web;
a first surface defining a garment facing side to the non-woven web;
a second surface defining a user facing side to the non-woven web;
wherein the second surface is separated from the first surface by a first distance;
a plurality of apertures extending through the non-woven web from the first surface to the second surface;
wherein the plurality of apertures is formed by pressing a plurality of pins, heated within a range of 150-240 °F (65.56-115.56 °C), through the non-woven web, causing the plurality of pins to punch through the non-woven web;
wherein the plurality of apertures define a plurality of proximal sides and distal sides;
wherein the proximal sides are located at the first surface;
wherein the distal sides define a third surface disposed from the first surface by a second distance;
wherein the second distance defines a loft of the non-woven web;
wherein the second distance is greater than the first distance; and
wherein the loft is greater than the first distance within a range of 180%-230%.

2. The pin punched apertured non-woven web according to claim 1, further comprising:
a plurality of bond points securing the plurality of fibers to one another to define the non-woven web.

3. The pin punched apertured non-woven web according to claim 1, wherein the plurality of bond points comprise at least one of ultrasonic welds, thermal bonds, pressure bonds, mechanically deformed bonds, resin bonds, adhesive bonds, and bonds between individual fibers.

4. The pin punched apertured non-woven web according to claim 1, wherein the distal sides of the plurality of apertures comprise edge portions that differ from ideal edge perimeters, defining an aperture fidelity within a range of 0.80-1.00, within a range of 0.85-0.95, within a range of 0.87-0.93, within a range of 0.89-0.91, or about 0.90.

5. The pin punched apertured non-woven web according to claim 1, wherein:
the non-woven web has a first open area without the plurality apertures and a second open area with the plurality of apertures;
the second open area is greater than the first open area; and
a ratio of the first open area to the second open area is within a range of 1.5-9.0, within a range of 4.0-5.0, within a range of 4.3-4.7, within a range of 4.4-4.6, or about 4.5.

6. The pin punched apertured non-woven web according to claim 1, wherein:
wherein the loft is greater than the first distance within a range of 190%-210%, within a range of 195%-205%, or about 200%.

7. The pin punched apertured non-woven web according to claim 1, wherein:
the plurality of fibers comprises polyethylene.

8. The pin punched apertured non-woven web according to claim 1, wherein:
the plurality of fibers comprises polyethylene and polyethylene terephthalate.

9. The pin punched apertured non-woven web according to claim 1, wherein:
the plurality of fibers comprises polypropylene.

10. The pin punched apertured non-woven web according to claim 1, wherein:
the plurality of fibers comprises polyethylene and polypropylene.

11. The pin punched apertured non-woven web according to claim 1, wherein:
the plurality of fibers comprises polyethylene and polylactic acid.

12. The pin punched apertured non-woven web according to claim 1, wherein:
the plurality of fibers comprises polyethylene terephthalate and co-polyethylene terephthalate.

13. The pin punched apertured non-woven web according to claim 1, wherein the plurality of fibers comprise a glass transition temperature between 5° and 85 °C (122-185 °F), wherein the plurality of fibers comprises at least one material.

14. The pin punched apertured non-woven web according to claim 1, wherein the plurality of fibers have a weight from 1 to 10 Den.

15. An absorbent product, comprising:
a pin punched apertured non-woven web;
an absorbent core disposed adjacent to the pin punched apertured non-woven web;
a back sheet disposed adjacent to the absorbent core, wherein the absorbent core is sandwiched between the pin punched apertured non-woven web and the backsheet,
wherein the pin punched apertured non-woven web comprises:
a plurality of fibers defining a non-woven web;
a first surface defining a garment facing side to non-woven web;
a second surface defining a user facing side to non-woven web;
wherein the second surface is separated from the first surface by a first distance;
a plurality of apertures extending through the non-woven web from the first surface to the second surface;
wherein the plurality of apertures is formed by pressing a plurality of pins, heated within a range of 150-240 °F (65.56-115.56 °C), through the non-woven web, causing the plurality of pins to punch through the non-woven web;
wherein the plurality of apertures define a plurality of proximal sides and distal sides;
wherein the proximal sides are located at the first surface;
wherein the distal sides define a third surface disposed from the first surface by a second distance;
wherein the second distance defines a loft of the non-woven web;
wherein the second distance is greater than the first distance; and
wherein the loft is greater than the first distance within a range of 180%-230%.

16. The absorbent product according to claim 15, further comprising:
a topsheet disposed adjacent to the pin punched apertured non-woven web, wherein the pin punched apertured non-woven web is sandwiched between the topsheet and the back sheet.

17. A pin punched apertured non-woven web, comprising:
a first non-woven web comprising
a plurality of fibers;
a first surface defining a garment facing side to the first non-woven web;
a second surface defining a user facing side to the first non-woven web;
wherein the second surface is separated from the first surface by a first distance;
a plurality of apertures extending through the first non-woven web from the first surface to the second surface;
wherein the plurality of apertures is formed by pressing a plurality of pins, heated within a range of 150-240 °F (65.56-115.56 °C), through the first non-woven web, causing the plurality of pins to punch through the first non-woven web;
wherein the plurality of apertures define a plurality of proximal sides and distal sides;
wherein the proximal sides are located at the first surface;
wherein the distal sides define a third surface disposed from the first surface by a second distance;
wherein the second distance defines a loft of the first non-woven web;
wherein the second distance is greater than the first distance; and
wherein the loft is greater than the first distance within a range of 180%-230%;
a second non-woven web comprising
a plurality of fibers;
a first surface defining a garment facing side to the second non-woven web;
a second surface defining a user facing side to the second non-woven web;
wherein the second surface is separated from the first surface by a first distance;
a plurality of apertures extending through the second non-woven web from the first surface to the second surface;
wherein the plurality of apertures is formed by pressing a plurality of pins, heated within a range of 150-240 °F (65.56-115.56 °C), through the second non-woven web, causing the plurality of pins to punch through the second non-woven web;
wherein the plurality of apertures define a plurality of proximal sides and distal sides;
wherein the proximal sides are located at the first surface;
wherein the distal sides define a third surface disposed from the first surface by a second distance;
wherein the second distance defines a loft of the first non-woven web;
wherein the second distance is greater than the first distance; and
wherein the loft is greater than the first distance within a range of 180%-230%,
wherein the first non-woven web is secured to the second non-woven web by at least the plurality of pins being punched therethrough.

18. The pin punched apertured non-woven web according to claim 17, wherein the first non-woven web is hydrophobic and the second non-woven web is hydrophilic.

19. The pin punched apertured non-woven web according to claim 17, wherein the first non-woven web is hydrophilic and the second non-woven web is hydrophobic.

20. An absorbent article, comprising:
the pin punched apertured non-woven web according to claim 17;
an absorbent core disposed adjacent to the pin punched apertured non-woven web; and
a back sheet disposed adjacent to the absorbent core, wherein the absorbent core is sandwiched between the pin punched apertured non-woven web and the backsheet.
